(19) United States
(12) Patent Application Publication
(10) Pub. No.: US 2005/0004065 A1
(43) Pub. Date: Jan. 6, 2005

(54) Hypoxia regulated genes

(75) Inventors: Elena Feinstein, Rehovot (IL); Paz Einat, Nes-Ziona (IL); Rami Skaliter, Nes-Ziona (IL)

Correspondence Address: BROWDY AND NEIMARK, P.L.L.C.;624 NINTH STREET, NW, SUITE 300, WASHINGTON, DC, 20001-5303, US

(73) Assignee: Quark Biotech, Inc., Pleasanton, CA (US)

(21) Appl. No.: 10/857,942

(22) Filed: Jun. 2, 2004

Related U.S. Application Data
(63) Continuation of application No. 09/997,263, filed on Nov. 30, 2001, now abandoned.
Application No. 09/997,263 is a continuation-in-part of application No. 09/802,806, filed on Mar. 9, 2001, now abandoned.
Application No. 09/802,806 is a continuation-in-part of application No. 09/384,446, filed on Aug. 27, 1999, now abandoned.
(60) Provisional application No. 60/098,158, filed on Aug. 27, 1998.
(60) Provisional application No. 60/132,684, filed on May 5, 1999.
(60) Provisional application No. 60/207,333, filed on May 30, 2000.

(51) Int. Cl. A61K 48/00; C12P 19/12; C12N 15/85

(52) U.S. Cl. 514/44; 435/455; 514/12

(57) ABSTRACT

The polynucleotide sequence of the 2-2-83 gene encodes the 2-2-83 protein. Hypoxic-associated pathologies may be regulated by administering an effective amount of a polynucleotide or protein of the present invention, or a direct or indirect biologically active product of enzymatic activity of the protein. Tumorigenesis may be inhibited by inhibiting the enzymatic activity of the protein of the present invention. The presence of a hypoxia-associated pathology may be diagnosed by screening for the reduced expression of the 2-2-83 gene.

### CROSS-REFERENCE TO RELATED APPLICATIONS

This application is a continuation of U.S. application Ser. No. 09/977,263, filed Nov. 30, 2001, now abandoned, which is a continuation-in-part of U.S. application Ser. No. 09/802,806, filed Mar. 9, 2001, now abandoned, which is a continuation-in-part of U.S. application Ser. No. 09/384,446, filed Aug. 27, 1999, now abandoned, which claims priority under 35 U.S.C. §119(e) from U.S. provisional application No. 60/098,158, filed Aug. 27, 1998, and of U.S. provisional application No. 60/132,684, filed May 5, 1999, all of which are hereby incorporated herein by reference. This application also claims priority under 35 U.S.C. §119(e) from U.S. provisional application No. 60/207,333, filed May 30, 2000, also incorporated by reference, of which said application Ser. No. 09/802,806 was filed as a non-provisional.

### BACKGROUND OF THE INVENTION

1. Technical Field

The present invention relates to the identification of the polynucleotide sequence of the 2-2-83 gene and the corresponding protein sequence in various mammals, including human, which gene is differentially expressed in several pathological systems, such as stroke, hypoxic retina and hypoxic regions of tumors.

2. Background Art

The level of tissue oxygenation plays an important role in normal development as well as in pathologic processes such as ischemia or tumorigenesis. Tissue oxygenation plays a significant regulatory/inducer role in both apoptosis and in angiogenesis (Bouck et al, 1996; Bunn et al, 1996; Dor et al, 1997; Carmeliet et al, 1998). Apoptosis (see Duke et al, 1996 for review) and growth arrest occur when cell growth and viability are reduced due to oxygen deprivation (hypoxia). Angiogenesis (i.e., blood vessel growth, vascularization) is stimulated when hypo-oxygenated cells secrete factors which stimulate proliferation and migration of endothelial cells in an attempt to restore oxygen homeostasis (for review see Hanahan et al, 1996).

Hypoxia plays a critical role in the selection of mutations that contribute to more severe tumorigenic phenotypes (Graeber et al, 1996). Identifying activated or inactivated genes and gene products in hypoxia and ischemia is needed.

Ischemic disease pathologies involve a decrease in the blood supply to a bodily organ, tissue or body part generally caused by constriction or obstruction of the blood vessels, as for example retinopathy, myocardial infarction and stroke. Therefore, apoptosis and/or angiogenesis as induced by the ischemic condition are also involved in these disease states. Neoangiogenesis is seen in some forms of retinopathy and in tumor growth. These processes are complex cascades of events controlled by many different genes reacting to the various stresses such as hypoxia.

The ability to monitor hypoxia-triggered activation of genes can provide a tool to identify not immediately evident ischemia in a patient. Identification of hypoxia-regulated genes permits the utilization of gene therapy or direct use of gene protein products or products of their activity (i.e., in the case of metabolic enzymes), or alternatively inactivation of target genes function for therapeutic intervention in treating the diseases and pathologies associated with hypoxia, ischemia and tumor growth.

Ischemia of the Brain. Brain injury such as trauma and stroke are among the leading causes of mortality and disability in the western world.

Traumatic brain injury (TBI) is one of the most serious reasons for hospital admission and disability in modern society. Clinical experience suggests that TBI may be classified into primary damage occurring immediately after injury, and secondary damage, which occurs during several days post injury. Current therapy of TBI is either surgical or else mainly symptomatic.

Cerebrovascular diseases occur predominately in the middle and late years of life. They cause approximately 200,000 deaths in the United States each year as well as considerable neurologic disability. The incidence of stroke increases with age and affects many elderly people, a rapidly growing segment of the population. These diseases cause either ischemia-infarction or intracranial hemorrhage.

Stroke is an acute neurologic injury occurring as a result of interrupted blood supply, resulting in an insult to the brain. Most cerebrovascular diseases present as the abrupt onset of focal neurologic deficit. The deficit may remain fixed, it may improve or progressively worsen, leading usually to irreversible neuronal damage at the core of the ischemic focus, whereas neuronal dysfunction in the penumbra may be treatable and or reversible. Prolonged periods of ischemia result in frank tissue necrosis. Cerebral edema follows and progresses over the subsequent 2 to 4 days. If the region of the infarction is large, the edema may produce considerable mass effect with all of its attendant consequences.

Neuroprotective drugs are being developed in an effort to rescue neurons in the penumbra from dying, though as yet none has been proven efficacious.

Damage to neuronal tissue can lead to severe disability and death. The extent of the damage is primarily affected by the location and extent of the injured tissue. Endogenous cascades activated in response to the acute insult play a role in the functional outcome. Efforts to minimize, limit and/or reverse the damage have the great potential of alleviating the clinical consequences.

### SUMMARY OF THE INVENTION

The present invention is based on the discovery of a gene, the expression of which is diminished by hypoxic conditions. This gene, designated as the 2-2-83 gene, having SEQ ID NO:1 or SEQ ID NO:3, expresses a protein, designated as the 2-2-83 protein, and having SEQ ID NO:2 or SEQ ID NO:4. Any naturally-occurring polynucleotide comprising either of SEQ ID NO:1 or SEQ ID NO:3 is comprehended by the present invention, as are naturally-occurring polynucleotides having at least 70% identity with such a sequence and, in naturally-occurring neural cells, has its expression decreased when the cells are subjected to neurotoxic stress. Related naturally-occurring polynucleotides which are capable of hybridizing under moderately stringent conditions to a naturally-occurring polynucleotide which includes SEQ ID NO:1 or SEQ ID NO:3 and, in naturally-occurring neural cells, have their expression decreased when the cells are subjected to neurotoxic stress, are also comprehended by the present invention. Fragments of any such naturally-occurring polynucleotides having at least 20 nucleotides and polynucleotide sequences complementary to any of the previously-mentioned polynucleotides are also comprehended by the present invention.

The present invention also comprehends a polypeptide which includes any protein which is encoded by a strand of a full-length cDNA within the scope of the naturally-occurring polynucleotides discussed above, as well as a variant of such polypeptides having an amino acid sequence with at least 70% identity thereto and that retains the biological activity thereof, or a fragment of such polynucleotide or variant which retains the biological activity thereof, or a functional derivative or salt of such polynucleotide or fragment. Such biological activity is preferably the enzymatic activity thereof. Preferably such polypeptide comprises the amino acid sequence of SEQ ID NO:2 or SEQ ID NO:4.

Molecules, such as antibodies, which comprise the antibody-binding portion of an antibody specific for such a polypeptide, variant or fragment are also comprehended by the present invention.

The 2-2-83 protein of the present invention has enzymatic activity and a molecule which is produced by cells expressing 2-2-83 and is present in the conditioned media thereof protects cells from oxidative stress and is also comprehended by the present invention. This molecule, which is the direct or indirect result of 2-2-83 activity, is a steroid.

The present invention is further related to pharmaceutical compositions comprising an effective amount of a polypeptide in accordance with the present invention, or the neuroprotective molecule present in the conditioned media of cells expressing 2-2-83, and a pharmaceutically acceptable excipient. The polypeptides or molecules of the present invention may be used to treat a subject suffering from oxidative stress. Such oxidative stress may that which results from stroke.

Such polypeptides and molecules are also useful for treating a subject suffering from a neurodegenerative disease or having a tendency to develop a neurodegenerative disease, such as Alzheimer's disease or Parkinson's disease.

The polypeptides and molecules of the present invention are also useful for alleviating or reducing damage to the central nervous system in a patient who has suffered an injury to the central nervous system, such as an ischemic episode. Such an ischemic episode may be a global or focal cerebral episode and may be secondary to a trauma to the central nervous system.

The present invention is further directed to methods of treating tumors by down-regulating the expression of the 2-2-83 gene or inhibiting the enzymatic activity of the 2-2-83 protein.

The present invention also comprehends methods for screening drugs that up-regulate or down-regulate a gene in accordance with the present invention. For example, cells transfected with and expressing DNA encoding the 2-2-83 gene may be contacted with a chemical compound to be screened. If the chemical compound up-regulates the 2-2-83 gene, it is identified as a potential up-regulating drug. If it down-regulates the 2-2-83 gene, it is identified as a potential down-regulating drug. Any potential up-regulating drug or down-regulating drug so identified may then be produced.

In addition, the present invention comprehends a method for diagnosing cells that have been subjected to hypoxia or ischemia by assaying for RNA having a sequence in accordance with the present invention or for the expression product thereof. The finding of down-regulation of such RNA or expression product as compared to a normal control indicates the likelihood that such cells have been subjected to hypoxia or ischemia.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a graph showing that incubation of 293-control cells in conditioned medium of 293-2-2-83 protects them from H₂O₂-mediated oxidative stress.

FIG. 2 is a graph showing the absence of protective effect against 24-hydroxycholesterol when a point mutation is present in 2-2-83.

FIG. 3 is a graph showing the increased proliferation rate of fresh 2-2-83 expressing C6 clones.

FIG. 4 is a graph showing the normalization of the proliferation rate of 2-2-83 expressing C6 clones following passaging in vitro.

FIGS. 5A and 5B are photographs showing the altered, more differentiated morphology of freshly infected BE2C cultures with pBABE/2-2-83.

FIGS. 6A and 6B are graphs showing the growth curves of two independent polyclonal BE2C cell populations expressing exogenous 2-2-83. FIG. 6A shows the growth curve of BE2C/2-2-83/pBABE cells; and FIG. 6B shows the growth curve of BE2C/pcDNA cells.

FIGS. 7A and 7B are graphs showing the sensitivity to chemical hypoxia of 2-2-83-expressing BE2C cells to hypoxia. FIG. 7A shows the sensitivity of 2-2-83/pBABE cells; and FIG. 7B shows the sensitivity of 2-2-83/pcDNA cells.

FIG. 8 is a graph showing the viability of C6 cells which overexpress 2-2-83 (as a percent of control) as function of concentration of 25-hydroxycholesterol.

FIG. 9 is a graph showing the viability of C6 cells which overexpress 2-2-83 (as a percent of control) as function of concentration of 24-hydroxycholesterol.

FIG. 10 is a graph showing the viability of C6 cells which overexpress 2-2-83 in the presence of 24-hydroxycholesterol after 24 or 48 hours of treatment.

FIG. 11 is a graph showing the viability of BE2C cells which overexpress 2-2-83 (as a percent of control) as a function of concentration of 24-hydroxycholesterol.

FIG. 12 is a graph showing the primary tumor weights of subcutaneously injected C6 transfected clones.

FIG. 13 is a graph showing the in vivo tumorigenicity of M4 transfected clones by measurement of in situ tumor growth following injection of the M4 transfected clones.

FIG. 14 is a graph showing the weight of spontaneous lung metastases of intrafootpad injected M4 transfected clones. The abbreviations identifying the clones are as set forth in FIG. 13.

FIG. 15 is a graph showing the weight of experimental lung metastases of intravenously injected M4 transfected clones.

FIG. 16 is a Northern blot analysis of stable PC12 clones expressing 2-2-83. Stable PC12 clones were prepared as described in the Materials and Methods section of Example 16. Total RNA was prepared and probed with 32P-labeled 2-2-83 specific probe. Lane 1=parental PC12 cells; Lane 2=pCDNA3-transfected cells; Lanes 3 and 4=pCDNA3/2-2-83 transfected clones 19 and 33, respectively.

FIG. 17 is a Northern blot analysis of inducible 2-2-83 expression in PC12 cells. PC12 clones expressing 2-2-83 in tetracycline-inducible manner were prepared as described in the Materials and Methods section of Example 16. The cells were grown in the presence or absence of tetracycline for 72 hours and total cell protein was analyzed by immunoblotting with 2-2-83 antibody followed by ECL.

FIG. 18 displays photographs showing 2-2-83 in NGF withdrawal model in neuronal PC12. Stable PC12 clones expressing 2-2-83 or control cells were differentiated into neuronal-like cells and deprived of NGF as described in the Materials and Methods section of Example 16. At 24 hours post-NGF withdrawal, the cell culture was photographed at 10× resolution.

FIG. 19 shows the expression of endogenous and exogenous 2-2-83 mRNA in wild type (WT) and transgenic (TG) mice. Total RNA was prepared from two independent lines of 2-2-83 TG and from WT FVB/N mice, blotted and probed with 2-2-83 specific probe. The filter was then exposed to X-ray film. Ht=Heart; Ctx=Cortex. Filled and hollow arrow heads indicate signal of endogenous and exogenous 2-2-83 mRNAs, respectively.

FIGS. 20A and 20B show that infarct volume is reduced in 2-2-83 transgenic mice in permanent MCAO model of brain ischemia. 2-2-83 TG FVB/N mice as well as their WT littermates were operated and the infarct was visualized 24 hours later as described in Example 18, materials and methods. The graphs shown represent data accumulated from two independent lines of mice. For WT, N=12; for TG, N=13. In FIG. 20A the infarct volume (V) was calculated for each slice as follows: V=(H*S)/3 where H is the slice thickness (300 μm) and S is the average area of the slice from both sides. The slices were numbered according to distance from core (position 0) and each unit on the X-axes equals to 300 μm. Negative and positive numbering refers to frontal and dorsal sides, respectively. FIG. 20B is the same as in FIG. 20A except V is presented as % of contralateral (right) hemisphere volume (measured as above).

### DETAILED DESCRIPTION OF THE INVENTION

I. Definitions

The following definitions apply to the terms used in the present specification and claims:

The term “gene” refers to the genomic nucleotide sequence which is transcribed to a full-length RNA. Such RNA molecules may be converted into corresponding cDNA molecules by techniques well known to the art of recombinant DNA technology. The term “gene” classically refers to the genomic sequence, which, upon processing, can produce different RNAs, e.g., by splicing events. However, for ease of reading, any full-length counterpart RNA sequence will also be referred to by shorthand herein as a “gene”.

The term “Expressed Sequence Tag” or “EST” refers to a partial DNA or cDNA sequence of about 150 to 500, more preferably about 300, sequential nucleotides of a longer sequence obtained from a genomic or cDNA library prepared from a selected cell, cell type, tissue type, organ or organism which longer sequence corresponds to an mRNA (or other full-length RNA) transcribed by a gene found in that library. In this case, the gene is found in rat neuronal cells. One or more libraries made from a single tissue type typically provide at least about 3,000 different (i.e., unique) ESTs and potentially the full complement of all possible ESTs representing all cDNAs, e.g., 50,000-100,000 in an animal such as a human. Further background and information on the construction of ESTs is described in Adams et al (1991) and International Application Number PCT/US92/05222 (Jan. 7, 1993).

The term “apoptosis” is particularly defined as the single deletion of scattered cells by fragmentation into membrane-bound particles which are phagocytosed by other cells, believed to be due to programmed cell death. However, as used herein, it should be understood that this term should be construed more broadly as encompassing neuronal cell death, whether or not that cell death is strictly by means the apoptotic process described above.

Two proteins are “cognate” if they are produced in different species, but are sufficiently similar in structure and biological activity to be considered the equivalent proteins for those species. Two proteins may also be considered cognate if they have at least 50% amino acid sequence identity (when globally aligned with a pam250 scoring matrix with a gap penalty of the form q+r(k−1) where k is the length of the gap, q=−12 and r=−4; percent identity=number of identities as percentage of length of shorter sequence) and at least one biological activity in common. Similarly, two genes are cognate if they are expressed in different species and encode cognate proteins.

II. Gene Discovery

Experiments were conducted to identify genes which are differentially modulated by hypoxia, i.e., genes which are suspected of either being induced by hypoxia after 16 hours, reduced by hypoxia after 16 hours, or induced by hypoxia after 4 hours, which genes are obtained either from the rat C6 glioma cell line or the human A172 glioma cell line. A microarray containing such genes was prepared. Each of the cell lines were exposed to hypoxic conditions (0.5% O₂ and 5% CO₂) for 4 or 16 hours and compared to cells grown under normal conditions (normoxia). Using the suppression subtractive hybridization (SSH) method, using the “PCR-Select cDNA Subtraction Kit” from Clontech, subtractive libraries were made, one of which was from normal vs. 16 hours hypoxia (genes reduced by hypoxia after 16 hours). 500 colonies were processed from the latter library in a gene microexpression microarray. The inserts of each plasmid were amplified by PCR and robotically fabricated on the glass. cDNA chip printing was performed by Synteni (Wang et al, 1999).
mRNA was then extracted from either C6 or A172 cells and labeled with fluorescent dNTP's using a reverse transcription reaction, using 50 μg template RNA (probes derived from nuclear RNA and total RNA), to generate a labeled cDNA probe. mRNA extracted from either C6 or A172 cells cultured in normoxic conditions were labeled with Cy3-dCTP (Amersham) and mRNA extracted from C6 or A172 cells cultured under hypoxic conditions were labeled with Cy5-dCTP (Amersham). The two labeled cDNA probes were then mixed and hybridized onto the microarrays (Schena et al, 1996; Wang et al, 1999). Following hybridization the microarray was scanned using a laser scanner and the amount of fluorescence of each of the fluorescent dyes was measured for each cDNA clone on the microarray, giving an indication of the level of mRNA in each of the original mRNA populations being tested. Comparison of the fluorescence on each cDNA clone on the microarray between the two different fluorescent dyes is a measure for the differential expression of the indicated genes between the two experimental conditions.

One candidate gene found following use of a probe from C6 or normoxia (Cy3 labeled) plus 16 hours hypoxia (Cy5 labeled) has been identified as gene 2-2-83 (sometimes referred to herein as 2-ii-83). The expression of this gene was found to be reduced after 16 hours of hypoxia, as compared to its amount of production under normal oxygen conditions.

The 2-2-83-specific cDNA probe hybridized to a single mRNA species of ˜4.0 Kb. Both rat and human orthologs of 2-2-83 cDNA were cloned. The rat and human nucleotide sequences are SEQ ID NOs:1 and 3, respectively, and their putative amino acid sequences are SEQ ID NOs:2 and 4, respectively. The rat cDNA clone is 3838 bp long and contains an open reading frame coding for a protein of 516 amino acids (nucl. 24-1572). The human cDNA is 4096 bp long and also codes for a 516 amino acid protein (nucl. 39-1587).

The human 2-2-83 nucleotide sequence is almost identical to human sequence D13643, designated as KIAA0018 (Nomura et al, 1994). However, the putative protein encoded by KIAA0018 cDNA (Q15392) appears truncated (390 amino acids instead of 516 acids encoded by human 2-2-83 gene). This is apparently due to a frame-shift mutation in this sequence or an error in the sequence which resulted in a deletion of the C residue between the positions 1166-1167. Accordingly, the full correct sequence of the 2-2-83 nucleotide sequence and protein amino acid sequence are novel. After the effective filing date of the present application, this same protein was published by others at Greeve et al (2000) under the name Seladin-1.

III. Expectations from Homologous Proteins

The putative proteins encoded by rat and human 2-2-83 genes are close homologs of protein 017397 from C. elegans and proteins found in several plant species (S71189 from Arabidopsis thaliana and P93472 from pea). The Arabidopsis thaliana homologue of gene 2-2-83 is a gene referred as diminuto (DIM) (Takahashi et al, 1995), dwarf1(DWF1) (Feldmann, 1991) or CBB1 (Kauschmann et al, 1996). The dim mutant was initially isolated as a slowly growing dwarf. This mutant phenotype could be rescued by the application of brassinolide (plant biologically active end pathway sterol). The comparison of sterol composition of normal and mutant plants has revealed that several biochemical reactions can be affected by dim mutation. In comparison to wild type plants, the mutant ones accumulate 24-methylenecholesterol and isofucosterol, but contain significantly reduced amounts of campesterol, sitosterol and end pathway sterols (Klahre et al, 1998). It was demonstrated that DIM activity is necessary for both the isomerization and reduction of 24-methylenecholesterol. DIM is an integral ER transmembrane protein that is anchored by its N-terminus into the membrane from the cytoplasmic side of the membranous compartment (Klahre et al, 1998).

As do animals, plants contain steroid compounds that are active at similarly low concentrations as steroid hormones. Animals mainly synthesize cholesterol; ergosterol is the predominant sterol in yeasts, and sitosterol, stigmasterol, and campesterol are the most abundant sterols in plants. In mammalian cells, cholesterol serves as the precursor of steroid hormones, which are characterized by reduced complexity caused by removal of most of the side chain. Plants use campesterol as a precursor for brassinosteroid (BR) biosynthesis and do not substantially shorten the side chain to form active hormones, but rather employ a series of reduction and hydroxylation steps to do so. BRs play an important role in plant growth and development. Over 60 analogues have been detected in a wide variety of plants in the past 18 years. Brassinolide is the most biologically active one. It elicits cell elongation/proliferation and shows strong synergistic interactions with auxin and additive interactions with gibberellins. Arabidopsis mutants that accumulate reduced endogenous amounts of BRs or BR-insensitive mutants have a very similar phenotype: they grow as dwarfs and their fertility is impaired (for reviews, see Clouse, 1996; McMorris, 1997). So far, three genes involved in brassinosteroid metabolism have been identified: CPD (Szerkeres et al, 1996), DWF4 (Choe et al, 1998), DET2 (Li et al, 1996) and one gene, BRI1 (receptor kinase), was shown to be involved in BR-mediated signal transduction (Li et al, 1997a). CPD, DWF4 and DET2 all encode cytochrome P450-like enzymes. DET2 encoding a close homolog of animal steroid 5a-reductase can also be substituted functionally, working on testosterone and progesterone as substrates (Li et al, 1997b).

Highly homologous proteins are likely to have similar functions. The mammalian 2-2-83 protein of the present invention is highly homologous to plant diminuto (44% identity). Another homologous protein is produced in C. elegans. C. elegans rely on plant sterols for their own sterol synthesis and are able to reduce 24-methylenencholesterol (Lozano et al, 1985). Therefore, the conservation of diminuto-like enzyme in this species is explainable. In yeast, no diminuto homologs were found. Accordingly, the reaction catalyzed by DIM presumably does not occur because the analogous reduction of the corresponding bond in ergosta-5,7,22,24(28)-tetraen-3-ol to yield ergosterol is known to be catalyzed by an unrelated enzyme. Using labeled precursors, the reaction catalyzed by DIM/DWF1 was not detected in mammalian cells (Nes et al, 1973). Therefore, the reason for the strict conservation of DIM sequences in animal cell evolution needs further study and explanation. There are three main possible explanations: (1) the reaction catalyzed by DIM is probably related only to the degradation of dietary sterols; (2) the reaction that is catalyzed by diminuto in animals is different from that in plants; (3) the reaction is similar, but the substrate is yet unknown. In general, there are few animal steroids possessing functionalized side chains, e.g., 25-hydroxy vitamin D3 (calcidiol); 1,25-dihydroxy vitamin D3 (calcitriol), their homologs, cholic acids and 24-oxy- (hydroxy-, epoxy-) steroids. The latter group of steroids emerged only recently in conjunction with biological activity. 24-hydroxy- and epoxysteroids are likely to activate the LXR receptors expressed in liver and brain. 24-oxysterol is highly abundant in brain. However, several reports indicate its potential neurotoxicity.

Tissue and cell-specific pattern of expression of gene 2-2-83 support its involvement in steroidogenesis in animals. Several sites where high levels of 2-2-83 mRNA were detected are known as sites where steroids are either synthesized or stored. Thus, 2-2-83 transcript was found in sebaceous glands where cholesterol compounds are among major constituents. In ovary, regulation of 2-2-83 expression did not correlate with the hypoxic state (judging by VEGF-specific staining), allowing the suggestion that it is related to steroidogenesis (estrogen and progesterone synthesis). In brain, expression of 2-2-83 was found mainly in brain stem and in the vicinity of spinal cord, regions rich in myelin that is also rich in steroids. Finally, expression of 2-2-83 in liver is connected to the synthesis of cholic acid.

In plants, DIM activity is crucial for cell elongation. In animals, cells that are able to elongate (to send long projections) are of neural and glial origin. Therefore, the function of gene 2-2-83 is somehow connected to neurite and axonal growth. Indeed, in rat brain, 2-2-83 is expressed in nuclei of brain stem, in reticular formation and in the stem-spinal cord boundary, all of which are the regions where neurons have extremely long projections. The fact of 2-2-83 expression in trophic oligodendrocytes points out that some final products of reactions catalyzed by the protein encoded by animal DIM possess neurotrophic and/or neuroprotective activity. Previously, several natural and synthetic steroids were found to have a neuroprotective activity. In this regard, it was found that down-regulation of 2-2-83-specific transcription was present in hypoxic retina. Significantly, 2-2-83 expression was enhanced compared to control 12 hours after the stroke when recovery processes may already start to take place. Since steroids synthesized with the aid of animal diminuto have neuroprotective activity, these compounds can be used as neuroprotective drugs at least in case of above-mentioned pathologies.

Because of its homology to the plant enzyme diminuto, as well as other evidence reported herein, it has been theorized that the protein encoded by the 2-2-83 gene is an enzyme.

The experiments reported herein establish that 2-2-83 preferentially expressed in normal cells that are involved in steroid synthesis. It protects from 24- and 25-OH-cholesterol toxicity in vitro. An anti-oxidative substance is secreted into the conditioned medium of 2-2-83 overexpressing cells. And a point mutation at a point similar to the point where a mutation causes dysfunction in the homologous plant protein, causes the mutant expressing cells to have no protection from 24-OH-cholesterol toxicity.

From all of these pieces of information, the present inventors now state that 2-2-83 is an enzyme involved in a hypoxia-regulated manner in the pathway of steroid synthesis which leads to a product, predicted to be a steroid, which is involved in cell proliferation, and also possibly in cell motility.

IV. Polynucleoxides

The present invention is directed to polynucleotide (nucleic acid) sequences whose expression is modulated by hypoxic conditions. More specifically, the polynucleotides are known as 2-2-83 with full-length sequences as set forth herein in SEQ ID NO:1 for the rat sequence and SEQ ID NO:3 for the cognate human sequence as well as analogs and fragments thereof.

The complete gene sequence of naturally-occurring variants of the 2-2-83 gene, such as, for example, allelic variations, may be determined by hybridization of a cDNA library using a probe which is based on the identified polynucleotide, under highly stringent conditions or under moderately stringent conditions. Stringency conditions are a function of the temperature used in the hybridization experiment and washes, the molarity of the monovalent cations in the hybridization solution and in the wash solution(s) and the percentage of formamide in the hybridization solution. In general, sensitivity by hybridization with a probe is affected by the amount and specific activity of the probe, the amount of the target nucleic acid, the detectability of the label, the rate of hybridization, and the duration of the hybridization. The hybridization rate is maximized at a Ti (incubation temperature) of 20-25° C. below Tm for DNA:DNA hybrids and 10-15° C. below Tm for DNA:RNA hybrids. It is also maximized by an ionic strength of about 1.5M Na⁺. The rate is directly proportional to duplex length and inversely proportional to the degree of mismatching.

Specificity in hybridization, however, is a function of the difference in stability between the desired hybrid and “background” hybrids. Hybrid stability is a function of duplex length, base composition, ionic strength, mismatching, and destabilizing agents (if any).

The Tm of a perfect hybrid may be estimated for DNA:DNA hybrids using the equation of Meinkoth et al (1984), as
Tm=81.5° C.+16.6 (logM)+0.41 (% GC)−0.61 (% form)−500/L
and for DNA:RNA hybrids, as
Tm=79.8° C.+18.5 (logM)+0.58 (% GC)−11.8 (% GC)²−0.56(% form)−820/L
where
M, molarity of monovalent cations, 0.01-0.4 M NaCl,
% GC, percentage of G and C nucleotides in DNA, 30%-75%,
% form, percentage formamide in hybridization solution, and
L, length hybrid in base pairs.

Tm is reduced by 0.5-1.5° C. (an average of 1° C. can be used for ease of calculation) for each 1% mismatching.

The Tm may also be determined experimentally. As increasing length of the hybrid (L) in the above equations increases the Tm and enhances stability, the full-length rat gene sequence can be used as the probe.

Filter hybridization is typically carried out at 68° C., and at high ionic strength (e.g., 5-6×SSC), which is non-stringent, and followed by one or more washes of increasing stringency, the last one being of the ultimately desired stringency. The equations for Tm can be used to estimate the appropriate Ti for the final wash, or the Tm of the perfect duplex can be determined experimentally and Ti then adjusted accordingly.

Hybridization conditions should be chosen so as to permit allelic variations, but avoid hybridizing to other genes. In general, stringent conditions are considered to be a Ti of 5° C. below the Tm of a perfect duplex, and a 1% divergence corresponds to a 0.5-1.5° C. reduction in Tm. Use of a Ti of 5-15° C. below, more preferably 5-10° C. below, the Tm of the double stranded form of the probe is recommended for probing a given cDNA library with EST probes of that species. However, when probing for a human gene cognate, more moderate stringency hybridization conditions should be used.

As used herein, highly stringent conditions are those which are tolerant of up to about 15% sequence divergence, while moderately stringent conditions are those which are tolerant of up to about 30-35% sequence divergence. Without limitation, examples of highly stringent (5-15° C. below the calculated Tm of the hybrid) and moderately stringent (15-30° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6× SSC (or 6× SSPE), 5× Denhardt's reagent, 0.5% SDS, 100 μg/ml denatured, fragmented salmon sperm DNA at an appropriate incubation temperature Ti.

Once any such naturally-occurring DNA is identified, it can be tested by means of routine experimentation to determine whether it is down-regulated in the cells in which it naturally occurs when subjected to hypoxic stress. The present invention is intended to comprehend any such naturally-occurring DNA which binds to SEQ ID NO:1 or 2 of the present invention or any oligonucleotide fragment thereof, preferably having at least 20, more preferably at least 50, contiguous nucleic acids, under highly stringent conditions or under moderately stringent conditions, which identified DNA molecules are determined to be down-regulated in the cells in which they naturally occur when such cells are subjected to hypoxic stress. Any such identified DNA molecules would be expected to have the same utility as discussed above for the identified polynucleotide.

V. Proteins and Polypeptides

The present invention is also directed to the proteins encoded by the polynucleotide sequences of the present invention and to polypeptides which are analogs, active fragments, functional derivatives and salts thereof.

Analogs of a protein or polypeptide encoded by the DNA sequences in accordance with the present invention are defined as follows. Preferably, the analog is a variant of the native sequence which has an amino acid sequence having at least 70% identity to the native amino acid sequence and retains the biological activity thereof. More preferably, such a sequence has at least 85% identity, at least 90% identity, or most preferably at least 95% identity to the native sequence.

The term “sequence identity” as used herein means that the sequences are compared as follows. The sequences are aligned using Version 9 of the Genetic Computing Group's GAP (global alignment program), using the default (BLOSUM62) matrix (values −4 to +11) with a gap open penalty of −12 (for the first null of a gap) and a gap extension penalty of −4 (per each additional consecutive null in the gap). After alignment, percentage identity is calculated by expressing the number of matches as a percentage of the number of amino acids in the claimed sequence.

Analogs in accordance with the present invention may also be determined in accordance with the following procedure. Polypeptides encoded by any nucleic acid, such as DNA or RNA, which hybridize to the complement of the native DNA or RNA under highly stringent or moderately stringent conditions, as long as that polypeptide maintains the biological activity of the native sequence are also considered to be within the scope of the present invention. Preferably, such nucleic acids hybridizing to the complement of the polynucleotides of the present invention under the specified conditions are naturally occurring nucleic acids, which may or may not be produced in cells of the same species as the original polynucleotides. As with any other analog, such polypeptide must retain the biological activity of the original polypeptide.

Stringency conditions are a function of the temperature used in the hybridization experiment, the molarity of the monovalent cations and the percentage of formamide in the hybridization solution. To determine the degree of stringency involved with any given set of conditions, one first uses the equation of Meinkoth et al (1984) for determining the stability of hybrids of 100% identity expressed as melting temperature Tm of the DNA-DNA hybrid:
Tm=81.5° C.+16.6 (LogM)+0.41 (% GC)−0.61 (% form)−500/L
where M is the molarity of monovalent cations, % GC is the percentage of G and C nucleotides in the DNA, % form is the percentage of formamide in the hybridization solution, and L is the length of the hybrid in base pairs. The Tm is reduced by 0.5-1.5° C. (an average of 1° C. can be used for ease of calculation) for each 1% mismatching. Thus, if the Tm used for any given hybridization experiment at the specified salt and formamide concentrations is 10° C. below the Tm calculated for a 100% hybrid according to equation of Meinkoth, hybridization will occur even if there is up to about 10% mismatch.

As used herein, highly stringent conditions are those which are tolerant of up to about 15% sequence divergence, while moderately stringent conditions are those which are tolerant of up to about 30-35% sequence divergence. Without limitation, examples of highly stringent (5-15° C. below the calculated Tm of the hybrid) and moderately (15-30° C. below the calculated Tm of the hybrid) conditions use a wash solution of 0.1×SSC (standard saline citrate) and 0.5% SDS at the appropriate Ti below the calculated Tm of the hybrid. The ultimate stringency of the conditions is primarily due to the washing conditions, particularly if the hybridization conditions used are those which allow less stable hybrids to form along with stable hybrids. The wash conditions at higher stringency then remove the less stable hybrids. A common hybridization condition that can be used with the highly stringent to moderately stringent wash conditions described above is hybridization in a solution of 6×SSC (or 6× SSPE), 5× Denhardt's reagent, 0.5% SDS, 100 μg/ml denatured, fragmented salmon sperm DNA at an appropriate incubation temperature Ti Tm. If mixed probes are used, it is preferable to use tetramethyl ammonium chloride (TMAC) instead of SSC (Ausubel, 1987, 1998).

The term “active fragments” is intended to cover any fragment of the proteins identified by means of the present invention that retain the biological activity of the full protein. For example, fragments can be readily generated from the full protein where successive residues can be removed from either or both the N-terminus or C-terminus of the protein, or from biologically active peptides obtained therefrom by enzymatic or chemical cleavage of the polypeptide. Thus, multiple substitutions are not involved in screening for active fragments. If the removal of one or more amino acids from one end or the other does not affect the biological activity, such as the enzymamtic activity, after testing in the standard tests, discussed herein, such truncated polypeptides are considered to be within the scope of the present invention. Further truncations can then be carried out until it is found where the removal of another residue destroys the biological activity.

“Functional derivatives” as used herein covers chemical derivatives which may be prepared from the functional groups which occur as side chains on the residues or the N- or C-terminal groups, by means known in the art, and are included in the invention as long as they remain pharmaceutically acceptable, i.e., they do not destroy the biological activity, such as the enzymatic activity, of the corresponding protein as described herein and do not confer toxic properties on compositions containing it. Derivatives may have chemical moieties, such as carbohydrate or phosphate residues, provided such a fraction has the same biological activity and remains pharmaceutically acceptable.

Suitable derivatives may include aliphatic esters of the carboxyl of the carboxyl groups, amides of the carboxyl groups by reaction with ammonia or with primary or secondary amines, N-acyl derivatives or free amino groups of the amino acid residues formed with acyl moieties (e.g., alkanoyl or carbocyclic aroyl groups) or O-acyl derivatives of free hydroxyl group (e.g., that of seryl or threonyl residues) formed with acyl moieties. Such derivatives may also include for example, polyethylene glycol side-chains which may mask antigenic sites and extend the residence of the complex or the portions thereof in body fluids.

Non-limiting examples of such derivatives are described below.

Cysteinyl residues most commonly are reacted with alpha-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, alpha-bromo-beta-(5-imidazoyl)propionic acid, chloroacetyl phosphate, B alkylmaleimides, 3-nitro-2-pyridyl disulfide, methyl-2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Parabromophenacyl bromide also is useful; the reaction is preferably performed in 0.1 M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2, 4-pentanedione; and transaminase-catalyzed reaction with glyoxylate.

Arginyl residues are modified by reaction with one or several conventional reagents, among them phenylglyoxal, 2,3-butanedione, 1,2-cyclodexanedione, and ninhydrin. Derivatization of arginine residues requires that the reaction be performed in alkaline conditions because of the high pKₐ of the guanidine functional group. Furthermore, these reagents may react with the groups of lysine as well as the arginine epsilon-amino group.

The specific modification of tyrosyl residues per se has been studied extensively, with particular interest in introducing spectral labels into tyrosyl residues by reaction with aromatic diazonium compounds or tetranitromethane. Most commonly, N-acetylimidazole and tetranitromethane are used to form O-acetyl tyrosyl species and 3-nitro derivatives, respectively.

Carboxyl side groups (aspartyl or glutamyl) are selectively modified by reaction with carbodiimides (R′—N—C—N—R′) such as 1-cyclohexyl-3-[2-morpholinyl-(4-ethyl)]carbodiimide or 1-ethyl-3-(4-azonia-4,4-dimethlypentyl) carbodiimide. Furthermore, aspartyl and glutamyl residues are converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

The term “functional derivatives” is intended to include only those derivatives that do not change one amino acid to another of the twenty commonly-occurring natural amino acids.

The term “salts” herein refers to both salts of carboxyl groups and to acid addition salts of amino groups of the complex of the invention or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must have substantially similar biological activity to the complex of the invention or its analogs.

VI. Product of Cellular Activity

As discussed hereinabove, the present inventors state that 2-2-83 protein is an enzyme which, by homology to the corresponding plant protein, is involved in steroid synthesis. It is known that the conditioned medium of cells which overexpress 2-2-83 has certain protective activity. The present invention comprehends that conditioned media and the active component thereof which confers protection from 24-hydroxycholesterol toxicity. It is predicted that this active component of the conditioned medium is a steroid either not previously known in humans or a known steroid with a previously unknown function, or a known protective steroid where regulation by 2-2-83 was previously unknown.

Once the reaction product of the 2-2-83 overexpressing cells is isolated, by means known in the art, and without undue experimentation, from the conditioned medium, its structure can be defined by NMR. Once the chemical structure is known, this product may be synthesized by well known methods starting from a typical steroid nucleus as is well known in the art. Once identified, this active component, which is expected to be a steroid, can be synthesized and will have utility, for example, in protecting the cells from the adverse effect of reactive oxygen and to ameliorate the effects of stroke.

VII. Utility

The experiments detailed in the present specification establish that there is utility in administering 2-2-83, up-regulating 2-2-83 gene expression, or administering the active component which is the result from the enzymatic process involving 2-2-83. For other indications, there is utility in inhibiting the expression of 2-2-83.

For example, overexpression of 2-2-83 has been shown to protect cells from oxidative stress. Furthermore, the experiments with hydrogen peroxide show that 2-2-83 expressing cells produce an anti-oxidative molecule which is secreted into the conditioned medium. Thus, an important utility of 2-2-83 is its involvement in the production of an active component having the pharmacological activity of protection from reactive oxygen damage and from toxicity by 24- and 25-hydroxycholesterol. This is an important utility for the 2-2-83 protein, or for pharmaceuticals which cause the up-regulation of the 2-2-83 gene, regardless of whether it is eventually determined that the protection from the damaging effects of 24- or 25-hydroxycholesterol is due to direct or indirect action of 2-2-83 on the cholesterol compounds.

In accordance with these findings, 2-2-83 or a related protein in accordance with the present invention, as well as the protective product thereof, which is in the conditioned media of cells overexpressing 2-2-83, or small molecules or peptides found in screens to up-regulate 2-2-83 expression or enhance its enzymatic activity, may be used for the treatment of stroke by the administration of a stroke-ameliorating or stroke-inhibiting amount of such an agent so as to at least partially prevent brain damage, or avert the occurrence or reduce the size and severity of an ischemic infarct due, for example, to stroke, aneurysm, cerebrovascular accident, apoplexy or other trauma.

The present invention, therefore, extends to methods or the treatment of stroke or other conditions caused or exacerbated by hypoxia or ischemia, and to corresponding pharmaceutical compositions, comprising and including, without limitation, as active ingredients, an agent as discussed above.

Within minutes after cessation of local cerebral blood flow, a region of densely ischemic brain tissue becomes infarcted and dies. This infarcted core is surrounded however, by a zone of ischemic but potentially viable tissue termed the “ischemic penumbra,” which receives suboptimal perfusion via collateral blood vessels. The volume of the penumbra that ultimately becomes infarcted after an acute arterial occlusion is determined by a variety of factors that mediate neurotoxicity within this zone during the hours following interrupted blood flow. The nature of these factors (including glutamate, superoxide radicals, and nitric oxide) is only partially understood, as are the complex interactions that will determine whether ischemic tissue will die or recover. Some of these factors are intrinsic to the locus of ischemia, and others are delivered to the penumbra via the circulation. The net result of signaling interactions between these factors can greatly enhance neuronal cytotoxicity in the ischemic penumbra, causing a significantly larger volume of brain damage and necrosis, with corresponding increases in functional damage. The agents of the present invention, participate in mediating increased volumes of cerebral infarction during focal cerebral ischemia.

The 2-2-83 gene may also be used as the target of screening processes to find agents capable of enhancing the expression thereof. Thus, the amount of the corresponding mRNA produced by a cell, before and after subjecting the cell to a neurotoxic stress, such as hypoxia, and administering a test agent, will determine whether that test agent causes further enhancement of expression of the 2-2-83 gene, as compared to a control in which no test agent is added. Such testing can reveal agents which are useful in the treatment of stroke. Screening methods are discussed in Section IX, hereinbelow.

The 2-2-83 gene may also be useful for diagnostic purposes. If in a tissue sample it is determined that the cells that normally produce 2-2-83 have lost this production, it is apparent that those cells have been subjected to hypoxia. Thus, this knowledge can be used in an assay to determine whether a given tissue sample has been subjected to hypoxia.

While 2-2-83 and its reaction product are beneficial for many purposes, it has also been found that 2-2-83 is overexpressed in many tumor cells. According to the properties demonstrated in other cell systems, 2-2-83 in tumor cells may contribute to tumorgenicity by positively regulating cell proliferation and by increasing resistance to oxidative stress. Thus, the inhibition of 2-2-83 in tumor cells is expected to be useful in diminishing the viability of such tumor cells. 2-2-83 expression in tumor cells may be inhibited by means of antisense technology, ribozyme technology or an application of a negative dominant polypeptide, as will be discussed in greater detail hereinbelow.

As 2-2-83 has also been-shown to be positive for the maintenance of neurons and to stimulate neuronal growth, another utility that is expected for this protein and its reaction product is in the treatment of neurodegenerative processes, e.g., degeneration occurring in either gray or white matter (or both) as a result of various diseases or disorders, including, without limitation: Alzheimer's disease, Parkinson's Disease, diabetic neuropathy, senile dementias, facial nerve (Bell's) palsy, glaucoma, Huntington's chorea, amyotrophic lateral sclerosis (ALS), non-arteritic optic neuropathy, intervertebral disc herniation, vitamin deficiency, prion diseases such as Creutzfeldt-Jakob disease, carpal tunnel syndrome, peripheral neuropathies associated with various diseases, including but not limited to, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyeloneuropathy, Giant axonal neuropathy, Refsum's disease, Fabry's disease, lipoproteinemia, etc. The 2-2-83 protein and related polypeptides in accordance with the present invention are also expected to be useful in the prevention or inhibition of secondary degeneration which may otherwise follow primary APL injury, e.g., blunt trauma, penetrating trauma, hemorrhagic stroke, ischemic stroke or damages caused by surgery such as tumor excision.

VIII. Diagnostic Methods

As the 2-2-83 gene of the present invention has been found to be modulated significantly downward after the cells have been subjected to hypoxia, such genes may be considered to be a gene of interest for the purpose of the diagnostic assays reported herein.

Methods of detecting tissue hypoxia in mammalian tissue are based on the use of the mRNA of the genes of interest or the translation product thereof as a diagnostic marker for cells that have been subjected to hypoxia or ischemia. It is possible to determine the level of the mRNAs or protein translation products corresponding to these bad genes, in normal tissue or bodily fluids as compared to hypoxic tissue a bodily fluid from a subject which has suffered a hypoxic event, and, thus, determine the reference values of these genes on mRNAs or proteins which are indicative of tissue hypoxia. For identification of the gene, in situ hybridization, Southern blotting, single strand conformational polymorphism, restriction endonuclease fingerprinting (REF), PCR amplification and DNA-chip analysis using the nucleic acid sequences of the present invention as probes/primers can be used.

Methods of obtaining tissue samples for analysis include any surgical and non-surgical technique known in the art. Surgical methods include, but are not limited to biopsy such as fine needle aspirate, core biopsy, dilation and curettage.

Samples. The sample for use in the detection methods may be of any biological fluid or tissue which is reasonably expected to contain the messenger RNA transcribed from one of the above genes of interest, or a protein expressed therefrom one of the above bad genes. The bodily fluids can include tears, serum, urine, sweat or other bodily fluid where secreted proteins from the tissue that is undergoing an ischemic event can be localized. Preferably, the sample is composed of cells from the subject being tested which are suspect of having been subjected to a hypoxic event, such as neural cells from a suspected stroke area or cardiac cells from a suspect infarct area.

Analyte Binding Reagents. The assay target or analyte as a diagnostic marker may be a nucleic acid, such as mRNA of a gene of interest, or a protein translation product thereof. When the assay target is a nucleic acid, the preferred binding reagent is a complementary nucleic acid. However, the nucleic acid binding agent may also be a peptide or protein. A peptide phage library may be screened for peptides which bind the nucleic acid assay target. In a similar manner, a DNA binding protein may be randomly mutagenized in the region of its DNA recognition site, and the mutants screened for the ability to specifically bind the target. Or the hypervariable regions of antibodies may be mutagenized and the antibody mutants displayed on phage.

When the assay target is a protein, the preferred binding reagent is an antibody, the specifically binding fragment of an antibody, or a molecule that has the antigen-binding portion of an antibody. The antibody may be monoclonal or polyclonal. It can be obtained by first immunizing a mammal with the protein target, and recovering either polyclonal antiserum, or immunocytes for later fusion to obtain hybridomas, or by constructing an antibody phage library and screening the antibodies for binding to the target. The binding reagent may also be a binding molecule other than an antibody, such as a receptor fragment, an oligopeptide, or a nucleic acid. A suitable oligopeptide or nucleic acid may be identified by screening a suitable random library.

Signal Producing System (SPS). In order to detect the presence, or measure the amount, of an analyte, the assay must provide for a signal producing system (SPS) in which there is a detectable difference in the signal produced, depending on whether the analyte is present or absent (or, in a quantitative assay, on the amount of the analyte). The detectable signal may be one which is visually detectable, or one detectable only with instruments. Possible signals include production of colored or luminescent products, alteration of the characteristics (including amplitude or polarization) of absorption or emission of radiation by an assay component or product, and precipitation or agglutination of a component or product. The term “signal” is intended to include the discontinuance of an existing signal, or a change in the rate of change of an observable parameter, rather than a change in its absolute value. The signal may be monitored manually or automatically.

Labels. The component of the signal producing system which is most intimately associated with the diagnostic reagent for the analyte is called the “label”. A label may be, e.g., a radioisotope, a fluorophore, an enzyme, a co-enzyme, an enzyme substrate, an electron-dense compound, an agglutinable particle, etc.

The radioactive isotope can be detected by such means as the use of a gamma counter or a scintillation counter or by autoradiography. Isotopes which are particularly useful for the purpose of the present invention are ³H, ³²P, ¹²⁵I, ¹³¹I, ³⁵S, and ¹⁴C.

Diagnostic kits are also within the scope of this invention. Such kits include monoclonal antibodies or nucleic acid probes that can rapidly detect tissue hypoxia.

For nucleic acid probes, the radioactive labeling can be carried out according to any conventional method such as terminal labeling at the 3′ or 5′ position with the use of a radiolabeled nucleotide, a polynucleotide kinase (with or without dephosphorylation by a phosphatase) or a ligase (according to the extremity to be labeled). The probes can be the matrix for the synthesis of a chain consisting of several radioactive nucleotides or of several radioactive and non-radioactive nucleotides. The probes can also be prepared by a chemical synthesis using one or several radioactive nucleotides. Another method for radioactive labeling is a chemical iodination of the probes of the invention which leads to the binding of several ¹²⁵I atoms on the probes.

The label may also be a fluorophore. When the fluorescently labeled reagent is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

Alternatively, fluorescence-emitting metals such as ¹²⁵Eu, or others of the lanthanide series, may be incorporated into a diagnostic reagent using such metal chelating groups as diethylenetriaminepentaacetic acid (DTPA) of ethylenediamine-tetraacetic acid (EDTA).

The label may also be a chemiluminescent compound. The presence of the chemiluminescently labeled reagent is then determined by detecting the presence of luminescence that arises during the course of a chemical reaction. Examples of particularly useful chemiluminescent labeling compounds are luminol, isolumino, theromatic acridinium ester, imidazole, acridinium salt and oxalate ester.

Likewise, a bioluminescent compound may be used for labeling. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Enzyme labels, such as horseradish peroxidase and alkaline phosphatase, can also be used. When an enzyme label is used, the signal producing system must also include a substrate for the enzyme. If the enzymatic reaction product is not itself detectable, the SPS will include one or more additional reactants so that a detectable product appears.

Conjugation Methods. A label may be conjugated, directly or indirectly (e.g., through a labeled anti-analyte binding reagent antibody), covalently (e.g., with N-succinimidyl 3-(2-pyridyldithio)propionate (SPDP)) or non-covalently, to the analyte binding reagent, to produce a diagnostic reagent.

Similarly, the analyte binding reagent may be conjugated to a solid phase support to form a solid phase (“capture”) diagnostic reagent.

Suitable supports include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention.

The support material may have virtually any possible structural configuration so long as the coupled molecule is capable of binding to its target. Thus the support configuration may be spherical, as in a bead, or cylindrical, as in the inside surface of a test tube, or the external surface of a rod. Alternatively, the surface may be flat such as a sheet, test strip, etc.

Binding Assay Formats. Binding assays may be divided into two basic types, heterogeneous and homogeneous. In heterogeneous assays, the interaction between the affinity molecule and the analyte does not affect the label, hence, to determine the amount or presence of analyte, bound label must be separated from free label. In homogeneous assays, the interaction does affect the activity of the label, and therefore analyte levels can be deduced without the need for a separation step.

In one embodiment, the analyte binding reagent is insolubilized by coupling it to a macromolecular support, and analyte in the sample is allowed to compete with a known quantity of a labeled or specifically labelable analyte analog. The “analyte analog” is a molecule capable of competing with analyte for binding to the analyte binding reagent, and the term is intended to include analyte itself. It may be labeled already, or it may be labeled subsequently by specifically binding the label to a moiety differentiating the analyte analogue from analyte. The solid and liquid phases are separated, and the labeled analyte analogue in one phase is quantified. The higher the level of analyte analogue in the solid phase, i.e., sticking to the analyte binding reagent, the lower the level of analyte in the sample.

In a “sandwich assay”, both an insolubilized analyte binding reagent, and a labeled analyte binding reagent are employed. The analyte is captured by the insolubilized analyte binding reagent and is tagged by the labeled analyte binding reagent, forming a ternary complex. The reagents may be added to the sample in either order, or simultaneously. The analyte binding reagents may be the same or different. The amount of labeled analyte binding reagent in the ternary complex is directly proportional to the amount of analyte in the sample.

The two embodiments described above are both heterogeneous assays. However, homogeneous assays are conceivable. The key is that the label be affected by whether or not the complex is formed.

Detection of Genes of Interest. Detection of the mRNA of the genes of interest may be done by Northern blot analysis on tissue biopsies. Tissue samples from patients may be obtained and the total RNA extracted using RNAStat 60. The total RNA sample may then be resolved on denaturing gel by electrophoresis and then transferred onto a nylon membrane. After transfer of RNA onto the membrane, the membrane may then be used in hybridization with a suitable probe, which may be a synthetic probe directed against a gene already known to be a marker, or which may be a cDNA probe prepared directly from subtractive hybridization, wherein the fragment encoding the gene of interest, that is up-regulated in tissue hypoxia, will be labeled, preferably either radioactively with ³²P or non-radioactively with DIG (Digoxigenin). A negative control, such as one composed of RNA sample from normal tissue of normal subjects, may be resolved side by side with the patients' sample, to determine quantitatively whether there is a significant increase in the level of gene expression. Elevation of the messenger RNA transcript from this gene would imply the presence of hypoxia, ischemia or other neurotoxic stress.

In a hybridization assay, a nucleic acid reagent is used as a probe. For probe use, only one reagent is needed, and it may hybridize to all or just a part of the target nucleic acid. Optionally, more than one probe may be used to increase specificity.

In probe-based assays, hybridizations may be carried out on filters or in solutions. Typical filters are nitrocellulose, nylon, and chemically-activated papers. The probe may be double stranded or single stranded, however, the double stranded nucleic acid will be denatured for binding.

Techniques for detecting a protein translation product of interest include, but are not limited to, immunoblotting or Western blotting, ELISA, sandwich assays, fluorescence, or biotin or enzymatic labeling with or without secondary antibodies.

Western blot analysis can be done on the tissue biopsies or tissue aspirates. This would involve resolving the proteins on an electrophoretic gel, such as an SDS PAGE gel, and transferring the resolved proteins onto a nitrocellulose or other suitable membrane. The proteins are incubated with a target binding molecule, such as an antibody.

This binding reagent may be labeled or not. If it is unlabeled, then one would also employ a secondary, labeled molecule which binds to the binding reagent. One approach involves avidinating one molecule and biotinylating the other. Another is for the secondary molecule to be a secondary antibody which binds the original binding reagent.

To improve detection of the specific protein, immunoprecipitation can be conducted. This typically will involve addition of a monoclonal antibody against the protein of interest to samples, then allowing the Ig-protein complex to precipitate after the addition of an affinity bead (i.e., antihuman Ig Sepharose bead). The immunoprecipitates will undergo several washings prior to transfer onto a nitrocellulose membrane. The Western blot analysis can be performed using another antibody against the primary antibody used.

There are a number of different methods of delivering the radiolabeled analyte binding reagent to the end-user in an amount sufficient to permit subsequent dynamic and/or static imaging using suitable radiodetecting devices. It may be administered by any means that enables the active agent to reach the agent's site of action in the body of a mammal. Because proteins and nucleic acids are subject to being digested when administered orally, parenteral administration, i.e., intravenous, subcutaneous, or intramuscular, would ordinarily be used to optimize absorption of an analyte binding reagent, such as an antibody, which is a protein.

The dosage is the smallest amount capable of providing a diagnostically effective image, and may be determined by means conventional in the art, using known radioimaging agents as a guide.

Typically, the imaging is carried out on the whole body of the subject, or on that portion of the body or organ relevant to the condition or disease under study. The amount of radiolabeled analyte binding reagent accumulated at a given point in time in relevant target organs can then be quantified.

A particularly suitable radiodetecting device is a scintillation camera, such as a gamma camera. A scintillation camera is a stationary device that can be used to image distribution of radiolabeled analyte binding reagent. The detection device in the camera senses the radioactive decay, the distribution of which can be recorded. Data produced by the imaging system can be digitized. The digitized information can be analyzed over time discontinuously or continuously. The digitized data can be processed to produce images, called frames, of the pattern of uptake of the radiolabeled analyte binding reagent in the target tissue/organ at a discrete point in time. In most continuous (dynamic) studies, quantitative data is obtained by observing changes in distributions of radioactive decay in the target tissue/organ over time. In other words, a time-activity analysis of the data will illustrate uptake through clearance of the radiolabeled binding protein by the target organs with time.

Various factors should be taken into consideration in selecting an appropriate radioisotope. The radioisotope must be selected with a view to obtaining good quality resolution upon imaging, should be safe for diagnostic use in humans and animals (except for animal models which will be sacrificed thereafter and will be maintained anaesthetized until then), and should preferably have a short physical half-life so as to decrease the amount of radiation received by the body (with the same exceptions). The radioisotope used should preferably be pharmacologically inert, and, in the quantities administered, should not have any substantial physiological effect.

The analyte binding reagent may be radiolabeled with different isotopes of iodine, for example ¹²³I, ¹²⁵I, or ¹³¹I (see for example, U.S. Pat. No. 4,609,725). The extent of radiolabeling must, however be monitored, since it will affect the calculations made based on the imaging results (i.e., a diiodinated analyte binding reagent will result in twice the radiation count of a similar monoiodinated analyte binding reagent over the same time frame).

In applications to human subjects, it may be desirable to use radioisotopes other than ¹²⁵I for labeling in order to decrease the total dosimetry exposure of the human, body and to optimize the detectability of the labeled molecule (though this radioisotope can be used if circumstances require). Ready availability for clinical use is also a factor. Accordingly, for human applications, preferred radiolabels are for example, ^{99m}Tc, ⁶⁷Ga, ⁶⁸Ga, ⁹⁰Y, ¹¹¹In, ^{113m}In ¹²³I, ¹⁸⁶Re, ¹⁸⁸Re or ²¹¹At.

The radiolabeled analyte binding reagent may be prepared by various methods. These include radiohalogenation by the chloramine-T method or the lactoperoxidase method and subsequent purification by HPLC (high pressure liquid chromatography), for example as described by Gutkowska et al (1987). Other known method of radiolabeling can be used, such as IODOBEADS™.

For animal models, such as mice or rats, the animal may be sacrificed after administration of the analyte binding reagent and regions which have been subjected to neurotoxic stress imaged on immobilized brain slices.

IX. Screening Methods

The 2-2-83 gene identified by means of the present invention, or a related gene in accordance with the present invention, can be used as a candidate gene in a screening assay for identifying and isolating small molecules or peptides which enhance the expression of 2-2-83 or related genes in accordance with the present invention or for enhancing the enzymatic and/or other biological activity of the 2-2-83 protein or related proteins in accordance with the present invention. Such genes may also be used as candidate genes in screening assays for identifying and isolating inhibitors of gene expression or of the enzymatic or other biological activity of the gene products.

Many types of screening assays are known to those of ordinary skill in the art. The specific assay which is chosen will depend to a great extent on the activity of the candidate gene being screened or of the protein expressed thereby. Thus, as the expression product of the 2-2-83 gene has enzymatic activity, an assay which is based on enhancement or inhibition of the enzymatic activity may be used. Even if the specific enzymic substrate is not known, the effect of the enzymatic process is known from the tests conducted in the various examples herein. Thus, any of these examples can be used in a screening assay to find enhancers or inhibitors of the end effect of that enzymatic or other biological activity.

If it is determined that the 2-2-83 protein binds to a ligand or other interactor, then the assay can be based on the inhibition of such binding or interaction.

As is well known in the art, the screening assays may be in vivo or in vitro. An in vivo assay is a cell-based assay using any eukaryotic cell. One such cell-based system is particularly relevant in order to directly measure the activity of candidate genes which are pro-apoptotic functional genes, i.e., expression of the gene will cause apoptosis or otherwise cause cell death in target cells. One way of running such an in vivo assay uses tetracycline-inducible (Tet-inducible) gene expression. Tet-inducible gene expression is well known in the art (Hofmann et al, 1996). Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, indicating that expression is regulated uniformly within the infected cell population.

When dealing with a specific biological function, Tet-inducible expression causes that function in target cells. One can screen for small molecules or peptides able to modulate that activity.

If the gene product of the candidate gene phosphorylates with a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced vs. non-induced genes provides a measure of reporter gene activation.

In a similar indirect assay, a reporter system can be designed that responds to changes in protein-protein interaction of the candidate protein. If the reporter responds to actual interaction with the candidate protein, a color reaction will occur.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter will be a direct assay of stimulation/inhibition of the reporter gene.

Various in vitro screening assays are also well within the skill of those of ordinary skill in the art. For example, if enzymatic activity is to be measured, the target protein can be defined and specific enzymatic reaction of the target can be followed. The assay may involve either inhibition of the enzymatic activity on the target or stimulation of enzymatic activity on the target, both types of assay being well known in the art.

One can also measure in vitro interaction of a candidate protein with interactors. In this screen, the candidate protein is immobilized on beads. An interactor, such as a receptor ligand, is radioactively labeled and added. When it binds to the candidate protein on the bead, the amount of radioactivity carried on the beads (due to interaction with the candidate protein) can be measured. The assay would indicate inhibition of the interaction by measuring the amount of radioactivity on the bead.

The target molecules, which may be used in the screens of the present invention, may be randomly-generated peptides or peptides that encompass the entire library of possible amino acid combinations. The molecules used in the screen may also include a variety of organic molecules, including drugs known for other indications. The broth of biological matter, such as bacteria culture products, fungi culture products, eukaryotic culture products, and crude cytokine preparations, may also be screened in the methods of the present invention described herein. There need be no expectation of effectivity going into the screen as it is the purpose of the screen to identify peptides and small molecules that would have an increased likelihood of being useable as a therapeutic agent or for further study in order to identify the ultimate therapeutic agents.

Any of the screening assays, according to the present invention, will include a step of identifying the small molecule or peptide which tests positive in the assay and may also include the further step of producing that which has been so identified. As the small molecule or peptide identified in the course of the screen is preexisting, once it has been identified, it can be readily synthesized or otherwise produced in purified form for further screening tests or for therapeutic use. The use of any such molecules so identified is also considered to be part of the present invention.

X. Antibodies

The present invention also comprehends antibodies specific for the 2-2-83 proteins. Such antibodies may be used for diagnostic purposes to identify the presence of any such naturally-occurring proteins. Such antibody may be a polyclonal antibody or a monoclonal antibody or any other molecule that incorporates the antigen-binding portion of a monoclonal antibody specific for such a protein. Such other molecules may be a single-chain antibody, a humanized antibody, an F(ab) fraction, a chimeric antibody, an antibody to which is attached a label, such as fluorescent or radioactive label, or an immunotoxin in which a toxic molecule is bound to the antigen binding portion of the antibody. The examples are intended to be non-limiting. However, as long as such a molecule includes the antigen-binding portion of the antibody, it will be expected to bind to the protein and, thus, can be used for the same diagnostic purposes for which a monoclonal antibody can be used.

Conveniently, the antibodies can be prepared against the immunogen or portion thereof for example a synthetic peptide based on the sequence, or prepared recombinantly by cloning techniques or the natural gene product and/or portions thereof can be isolated and used as the immunogen. Immunogens can be used to produce antibodies by standard antibody production technology well known to those skilled in the art as described generally in Harlow et al (1988) and Borrebaeck (1992). Antibody fragments can also be prepared from the antibodies and include Fab, F(ab′)₂, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

For producing monoclonal antibodies the technique involves hyperimmunization of an appropriate donor with the immunogen, generally a mouse, and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

For producing recombinant antibody (see generally Huston et al, 1991; Johnson et al, 1991; Mernaugh et al, 1995), messenger RNAs from antibody producing B-lymphocytes of animals, or hybridoma are reverse-transcribed to obtain complimentary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated, as is well known in the art. (For a general discussion of conjugation of fluorescent or enzymatic moieties see Johnstone et al, 1982.) The binding of antibodies to a solid support substrate is also well known in the art. (See for a general discussion Harlow et al, 1988, and Borrebaeck, 1992). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹⁴C and iodination.

XI. Antisense Sequences

In order to manipulate the expression of the 2-2-83 protein, it is desirable to produce antisense RNA in a cell. To this end, the complete or partial cDNA of 2-2-83 is inserted into an expression vector comprising a promoter. The 3′ end of the cDNA is thereby inserted adjacent to the 3′ end of the promoter, with the 5′ end of the cDNA being separated from the 3′ end of the promoter by said cDNA. Upon expression of the cDNA in a cell, an antisense RNA is therefore produced which is incapable of coding for the protein. The presence of antisense RNA in the cell reduces the expression of the cellular (genomic) copy of the bad gene.

For the production of antisense RNA, the complete cDNA may be used. Alternatively, a fragment thereof may be used, which is preferably between about 9 and 2,000 nucleotides in length, more preferably between 15 and 500 nucleotides, and most preferably between 30 and 150 nucleotides.

The fragment is preferably corresponding to a region within the 5′ half of the cDNA, more preferably the 5′ region comprising the 5′ untranslated region and/or the first exon region, and most preferably comprising the ATG translation start site. Alternatively, the fragment may correspond to DNA sequence of the 5′ untranslated region only.

Antisense intervention in the expression of specific genes can be achieved by the use of synthetic AS oligonucleotide sequences (for recent reports see Lefebvre-d'Hellencourt et al, 1995; Agrawal, 1996; Lev-Lehman et al, 1997). The oligonucleotide is preferably a DNA oligonucleotide. The length of the antisense oligonucleotide is preferably between 9 and 150, more preferably between 12 and 60, and most preferably between 15 and 50 nucleotides. Suitable antisense oligonucleotides that inhibit the production of the protein of the present invention from its encoding mRNA can be readily determined with only routine experimentation through the use of a series of overlapping oligonucleotides similar to a “gene walking” technique that is well-known in the art. Such a “walking” technique as well-known in the art of antisense development can be done with synthetic oligonucleotides to walk along the entire length of the sequence complementary to the mRNA in segments on the order of 9 to 150 nucleotides in length. This “gene walking” technique will identify the oligonucleotides that are complementary to accessible regions on the target mRNA and exert inhibitory antisense activity.

The AS oligonucleotide sequence is designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, 1996). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive.

The sequence target segment for the antisense oligonucleotide is selected such that the sequence exhibits suitable energy related characteristics important for oligonucleotide duplex formation with their complementary templates, and shows a low potential for self-dimerization or self-complementation (Anazodo et al, 1996). For example, the computer program OLIGO 4.0 (National Biosciences, Inc.), can be used to determine antisense sequence melting temperature, free energy properties, and to estimate potential self-dimer formation and self-complimentary properties. The program allows the determination of a qualitative estimation of these two parameters (potential self-dimer formation and self-complimentary) and provides an indication of “no potential” or “some potential” or “essentially complete potential”. Using this program target segments are generally selected that have estimates of no potential in these parameters. However, segments can be used that have “some potential” in one of the categories. A balance of the parameters is used in the selection as is known in the art. Further, the oligonucleotides are also selected as needed so that analog substitution do not substantially affect function.

Alternatively, an oligonucleotide based on the coding sequence of a protein capable of binding to a 2-2-83 or the protein encoded thereby can be designed using Oligo 4.0 (National Biosciences, Inc.). Antisense molecules may also be designed to inhibit translation of an mRNA into a polypeptide by preparing an antisense which will bind in the region spanning approximately −10 to +10 nucleotides at the 5′ end of the coding sequence.

The mechanism of action of antisense RNA and the current state of the art on use of antisense tools is reviewed in Kumar et al (1998). There are reviews on the chemical (Crooke, 1995; Uhlmann et al, 1990), cellular (Wagner, 1994) and therapeutic (Hanania, et al, 1995; Scanlon, et al, 1995; Gewirtz, 1993) aspects of this rapidly developing technology. The use of antisense oligonucleotides in inhibition of BMP receptor synthesis has been described by Yeh et al (1998). The use of antisense oligonucleotides for inhibiting the synthesis of the voltage-dependent potassium channel gene Kv1.4 has been described by Meiri et al (1998). The use of antisense oligonucleotides for inhibition of the synthesis of Bcl-x has been described by Kondo et al (1998). The therapeutic use of antisense drugs is discussed by Stix (1998); Flanagan (1998); Guinot et al (1998), and references therein. Within a relatively short time, ample information has accumulated about the in vitro use of AS nucleotide sequences in cultured primary cells and cell lines as well as for in vivo administration of such nucleotide sequences for suppressing specific processes and changing body functions in a transient manner. Further, enough experience is now available in vitro and in vivo in animal models and human clinical trials to predict human efficacy.

Modifications of oligonucleotides that enhance desired properties are generally used when designing antisense oligonucleotides. For instance, phosphorothioate bonds are used instead of the phosphoester bonds that naturally occur in DNA, mainly because such phosphorothioate oligonucleotides are less prone to degradation by cellular enzymes. Peng Ho et al teach that undesired in vivo side effects of phosphorothioate oligonucleotides may be reduced when using a mixed phosphodiester-phosphorothioate backbone. Preferably, 2′-methoxyribonucleotide modifications in 60% of the oligonucleotide is used. Such modified oligonucleotides are capable of eliciting an antisense effect comparable to the effect observed with phosphorothioate oligonucleotides. Peng Ho et al teach further that oligonucleotide analogs incapable of supporting ribonuclease H activity are inactive.

Therefore, the preferred antisense oligonucleotide of the present invention has a mixed phosphodiester-phosphorothioate backbone. Preferably, 2′-methoxyribonucleotide modifications in about 30% to 80%, more preferably about 60%, of the oligonucleotide are used.

In the practice of the invention, antisense oligonucleotides or antisense RNA may be used. The length of the antisense RNA is preferably from about 9 to about 3,000 nucleotides, more preferably from about 20 to about 1,000 nucleotides, most preferably from about 50 to about 500 nucleotides.

In order to be effective, the antisense oligonucleotides of the present invention must travel across cell membranes. In general, antisense oligonucleotides have the ability to cross cell membranes, apparently by uptake via specific receptors. As the antisense oligonucleotides are single-stranded molecules, they are to a degree hydrophobic, which enhances passive diffusion through membranes. Modifications may be introduced to an antisense oligonucleotide to improve its ability to cross membranes. For instance, the oligonucleotide molecule may be linked to a group which includes partially unsaturated aliphatic hydrocarbon chain and one or more polar or charged groups such as carboxylic acid groups, ester groups, and alcohol groups. Alternatively, oligonucleotides may be linked to peptide structures, which are preferably membranotropic peptides. Such modified oligonucleotides penetrate membranes more easily, which is critical for their function and may, therefore, significantly enhance their activity. Palmityl-linked oligonucleotides have been described by Gerster et al (1998). Geraniol-linked oligonucleotides have been described by Shoji et al (1998). Oligonucleotides linked to peptides, e.g., membranotropic peptides, and their preparation have been described by Soukchareun et al (1998). Modifications of antisense molecules or other drugs that target the molecule to certain cells and enhance uptake of the oligonucleotide by said cells are described by Wang (1998).

The antisense oligonucleotides of the invention are generally provided in the form of pharmaceutical compositions. These compositions are for use by injection, topical administration, or oral uptake.

Preferred uses of the pharmaceutical compositions of the invention by injection are subcutaneous injection, intraperitoneal injection, and intramuscular injection.

The pharmaceutical composition of the invention generally comprises a buffering agent, an agent which adjusts the osmolarity thereof, and optionally, one or more carriers, excipients and/or additives as known in the art, e.g., for the purposes of adding flavors, colors, lubrication, or the like to the pharmaceutical composition.

Carriers may include starch and derivatives thereof, cellulose and derivatives thereof, e.g., microcrystalline cellulose, Xanthum gum, and the like. Lubricants may include hydrogenated castor oil and the like.

A preferred buffering agent is phosphate-buffered saline solution (PBS), which solution is also adjusted for osmolarity.

A preferred pharmaceutical formulation is one lacking a carrier. Such formulations are preferably used for administration by injection, including intravenous injection.

The preparation of pharmaceutical compositions is well known in the art and has been described in many articles and textbooks, see e.g., Remington's Pharmaceutical Sciences, especially pp 1521-1712 therein (Gennaro, 1990).

Additives may also be selected to enhance uptake of the antisense oligonucleotide across cell membranes. Such agents are generally agents that will enhance cellular uptake of double-stranded DNA molecules. For instance, certain lipid molecules have been developed for this purpose, including the transfection reagents DOTAP (Boehringer Mannheim), Lipofectin, Lipofectam, and Transfectam, which are available commercially. For a comparison of various of these reagents in enhancing antisense oligonucleotide uptake, see e.g., Quattrone et al (1995) and Capaccioli et al (1993). The antisense oligonucleotide of the invention may also be enclosed within liposomes. The preparation and use of liposomes, e.g., using the above-mentioned transfection reagents, is well known in the art. Other methods of obtaining liposomes include the use of Sendai virus or of other viruses. Examples of publications disclosing oligonucleotide transfer into cells using the liposome technique are, e.g., Meyer et al (1998), Kita et al (1999), Nakamura et al (1998), Abe et al (1998), Soni et al (1998), Bai et al (1998), see also discussion in the same Journal p. 819-20, Bochot et al (1998), Noguchi et al (1998), Yang et al (1998), Kanamaru et al (1998), and references therein. The use of Lipofectin in liposome-mediated oligonucleotide uptake is described in Sugawa et al (1998). The use of fusogenic cationic-lipid-reconstituted influenza-virus envelopes (cationic virosomes) is described in Waelti et al (1998).

The above-mentioned cationic or non-ionic lipid agents not only serve to enhance uptake of oligonucleotides into cells, but also improve the stability of oligonucleotides that have been taken up by the cell.

XII. Ribozymes

Instead of an antisense sequence as discussed herein above, ribozymes can be utilized. This is particularly necessary in cases where antisense therapy is limited by stoichiometric considerations (Sarver et al, 1990). Ribozymes can then be used that will target the same sequence. Ribozymes are RNA molecules that possess RNA catalytic ability (see Cech for review) that cleave a specific site in a target RNA. The number of RNA molecules that are cleaved by a ribozyme is greater than the number predicted by stochiochemistry (Hampel et al, 1989; Uhlenbeck, 1987).

Given the known mRNA sequence of a gene, ribozymes, which are RNA molecule that specifically bind and cleave said mRNA sequence (see, e.g., Chen et al, 1992; Zhao et al, 1993; Shore et al, 1993; Joseph et al, 1993; Shimayama et al, 1993; and Cantor et al, 1993) may be designed.

Ribozymes catalyze the phosphodiester bond cleavage of RNA. Several ribozyme structural families have been identified including Group I introns, RNase P, the hepatitis delta virus ribozyme, hammerhead ribozymes and the hairpin ribozyme originally derived from the negative strand of the tobacco ringspot virus satellite RNA (sTRSV) (Sullivan, 1994; U.S. Pat. No. 5,225,347, columns 4-5). The latter two families are derived from viroids and virusoids, in which the ribozyme is believed to separate monomers from oligomers created during rolling circle replication (Symons, 1989 and 1992). Hammerhead and hairpin ribozyme motifs are most commonly adapted for trans-cleavage of mRNAs for gene therapy (Sullivan, 1994). The ribozyme type utilized in the present invention is selected as is known in the art. Hairpin ribozymes are now in clinical trial and are the preferred type. In general the ribozyme is from 30-100 nucleotides in length.

Accordingly, a ribozyme-encoding RNA sequence may be designed that cleaves the mRNA of a bad gene of the present invention. The site of cleavage is preferably located in the coding region or in the 5′ non-translated region, more preferably, in the 5′ part of the coding region close to the AUG translational start codon.

A DNA encoding a ribozyme according to the present invention may be introduced into cells by way of DNA uptake, uptake of modified DNA (see modifications for oligonucleotides and proteins that result in enhanced membrane permeability, as described above for oligonucleotides and described below for proteins), or viral vector-mediated gene transfer.

XIII. Negative Dominant Peptides

Negative dominant peptide refers to a peptide encoded by a cDNA sequence that encodes only a part of a protein, i.e. a peptide (see Herskowitz, 1987). This peptide can have a different function from the protein it was derived from. It can interact with the full protein and inhibit its activity or it can interact with other proteins and inhibit their activity in response to the full protein. Negative dominant means that the peptide is able to overcome the natural proteins and fully inhibit their activity to give the cell a different characteristic, such as resistance or sensitization to killing. For therapeutic intervention either the peptide itself is delivered as the active ingredient of a pharmaceutical composition or the cDNA can be delivered to the cell utilizing the same methods as for antisense delivery.

XIV. Virus-Mediated Cellular Targeting

The proteins, peptides and antisense sequences of the present invention may be introduced into cells by the use of a viral vector. The use of a vaccinia vector for this purpose is described in Chapter 16 of Ausubel et al (1994-2000). The use of adenovirus vectors has been described, e.g., by Teoh et al (1998), Narumi et al (1998), Pederson et al (1998), Guang-Lin et al (1998), and references therein, Nishida et al (1998), Schwarzenberger et al (1998), and Cao et al (1998). Retroviral transfer of antisense sequences has been described by Daniel et al (1998). The use of SV-40 derived viral vectors and SV-40 based packaging systems has been described by Fang et al (1997). The use of papovaviruses which specifically target B-lymphocytes, has been described by Langner et al (1998).

When using viruses as vectors, the viral surface proteins are generally used to target the virus. As many viruses, such as the above adenovirus, are rather unspecific in their cellular tropism, it may be desirable to impart further specificity by using a cell-type or tissue-specific promoter. Griscelli et al (1998) teach the use of the ventricle-specific cardiac myosin light chain 2 promoter for heart-specific targeting of a gene whose transfer is mediated by adenovirus.

Alternatively, the viral vector may be engineered to express an additional protein on its surface, or the surface protein of the viral vector may be changed to incorporate a desired peptide sequence. The viral vector may thus be engineered to express one or more additional epitopes which may be used to target said viral vector. For instance, cytokine epitopes, MHC class II-binding peptides, or epitopes derived from homing molecules may be used to target the viral vector in accordance with the teaching of the invention. The above Langer et al (1998) reference teach the use of heterologous binding motifs to target B-lymphotrophic papoaviruses.

XV. Pharmaceutical Compositions

The pharmaceutical compositions of the invention are prepared generally as known in the art. Thus, pharmaceutical compositions comprising nucleic acids, e.g., ribozymes, antisense RNA or antisense oligonucleotides, are prepared as described above for pharmaceutical compositions comprising oligonucleotides and/or antisense RNA. The above considerations apply generally also to other pharmaceutical compositions. For instance, the pharmaceutical composition of the invention may contain naked DNA, e.g., good genes or fragments or derivatives thereof and a pharmaceutically acceptable carrier as known in the art. A variety of ways to enhance uptake of naked DNA is known in the art. For instance, cationic liposomes (Yotsuyanagi et al, 1998), dicationic amphiphiles (Weissig et al, 1998), fusogenic liposomes (Mizuguchi et al, 1996), mixtures of stearyl-poly(L-lysine) and low density lipoprotein (LDL), terplex (Kim et al, 1998), and even whole bacteria of an attenuated mutant strain of Salmonella typhimurium (Paglia et al, 1998) have been used in the preparation of pharmaceutical compositions containing DNA.

Administration of virus particles has been described in prior art publications, see, e.g., U.S. Pat. No. 5,882,877, where Adenovirus based vectors and administration of the DNA thereof is described. The viral DNA was purified on a CsCl gradient and then dialyzed against Tris-buffered saline to remove CsCl. In these preparations, viral titers (pfu/ml) of 10¹⁴ to 10¹⁰ are preferably used. Administration of virus particles as a solution in buffered saline, to be preferably administered by subcutaneous injection, is known from U.S. Pat. No. 5,846,546. Croyle and coworkers (Croyle et al, 1998) describe a process for the preparation of a pharmaceutical composition of recombinant adenoviral vectors for oral gene delivery, using CsCl gradients and lyophilization in a sucrose-containing buffer.

The active ingredients of the pharmaceutical composition can include oligonucleotides that are nuclease resistant needed for the practice of the invention or a fragment thereof shown to have the same effect targeted against the appropriate sequence(s) and/or ribozymes. Combinations of active ingredients as disclosed in the present invention can be used including combinations of antisense sequences.

Where the pharmaceutical composition of the invention includes a polypeptide or protein According to the present invention, the composition will generally contain salts, preferably in physiological concentration, such as PBS (phosphate-buffered saline), or sodium chloride (0.9% w/v), and a buffering agent, such as phosphate buffer in water or in the well-known PBS buffer. In the following section, the term “polypeptide” is meant to include all proteins or peptides according to the invention. The preparation of pharmaceutical compositions is well known in the art, see e.g., U.S. Pat. Nos. 5,736,519, 5,733,877, 5,554,378, 5,439,688, 5,418,219, 5,354,900, 5,298,246, 5,164,372, 4,900,549, 4,755,383, 4,639,435, 4,457,917, and 4,064,236.

The polypeptide of the present invention, or a pharmacologically acceptable salt thereof is preferably mixed with an excipient, carrier, diluent, and optionally, a preservative or the like, pharmacologically acceptable vehicles as known in the art, see, e.g., the above U.S. patents. Examples of excipients include, glucose, mannitol, inositol, sucrose, lactose, fructose, starch, corn starch, microcrystalline cellulose, hydroxypropylcellulose, hydroxypropyl-methylcellulose, polyvinylpyrrolidone and the like. Optionally, a thickener may be added, such as a natural gum, a cellulose derivative, an acrylic or vinyl polymer, or the like.

The pharmaceutical composition is provided in solid, liquid or semi-solid form. A solid preparation may be prepared by blending the above components to provide a powdery composition. Alternatively, the pharmaceutical composition is provided as a lyophilized preparation. The liquid preparation is provided preferably as an aqueous solution, aqueous suspension, oil suspension or microcapsule composition. A semi-solid composition is provided preferably as hydrous or oily gel or ointment. About 0.001 to 60 w/v %, preferably about 0.05 to 25 w/v % of polypeptide is provided in the composition.

A solid composition may be prepared by mixing an excipient with a solution of the polypeptide of the invention, gradually adding a small quantity of water, and kneading the mixture. After drying, preferably in vacuo, the mixture is pulverized. A liquid composition may be prepared by dissolving, suspending or emulsifying the polypeptide of the invention in water, a buffer solution or the like. An oil suspension may be prepared by suspending or emulsifying the polypeptide of the invention in an oleaginous base, such as sesame oil, olive oil, corn oil, soybean oil, cottonseed oil, peanut oil, lanolin, petroleum jelly, paraffin, Isopar, silicone oil, fatty acids of 6 to 30 carbon atoms or the corresponding glycerol or alcohol esters. Buffers include Sorensen buffer (Ergeb Physiol, 12:393, 1912), Clark-Lubs buffer (J Bact, 2 (1):109, 191, 1917), Macllvaine buffer (J Biol Chem, 49:183, 1921), Michaelis buffer (Die Wasserstoffinonenkonzentration, p. 186, 1914), and Kolthoff buffer (Biochem Z, 179:410, 1926).

A composition may be prepared as a hydrous gel, e.g., for transnasal administration. A hydrous gel base is dissolved or dispersed in aqueous solution containing a buffer, and the polypeptide of the invention, and the solution warmed or cooled to give a stable gel.

Preferably, the polypeptide of the invention is administered through intravenous, intramuscular or subcutaneous administration. Oral administration is expected to be less effective, because the polypeptide may be digested before being taken up. Of course, this consideration may apply less to a polypeptide of the invention which is modified, e.g., by being a cyclic polypeptide, by containing non-naturally occurring amino acids, such as D-amino acids, or other modifications which enhance the resistance of the polypeptide to biodegradation. Decomposition in the digestive tract may be lessened by use of certain compositions, for instance, by confining the polypeptide of the invention in microcapsules such as liposomes. The pharmaceutical composition of the invention may also be administered to other mucous membranes. The pharmaceutical composition is then provided in the form of a suppository, nasal spray or sublingual tablet. The dosage of the polypeptide of the invention may depend upon the condition to be treated, the patient's age, bodyweight, and the route of administration, and will be determined by the attending physician.

The uptake of a polypeptide of the invention may be facilitated by a number of methods. For instance, a non-toxic derivative of the cholera toxin B subunit, or of the structurally related subunit B of the heal-labile enterotoxin of enterotoxic Escherichia coli may be added to the composition, see U.S. Pat. No. 5,554,378.

In another embodiment, the polypeptide of the invention is provided in a pharmaceutical composition comprising a biodegradable polymer selected from poly-1,4-butylene succinate, poly-2,3-butylene succinate, poly-1,4-butylene fumarate and poly-2,3-butylene succinate, incorporating the polypeptide of the invention as the pamoate, tannate, stearate or palmitate thereof. Such compositions are described, e.g., in U.S. Pat. No. 5,439,688.

In a further embodiment, a composition of the invention is a fat emulsion. The fat emulsion may be prepared by adding to a fat or oil about 0.1-2.4 w/w of emulsifier such as a phospholipid, an emulsifying aid, a stabilizer, mixing mechanically, aided by heating and/or removing solvents, adding water and isotonic agent, and optionally, adjusting adding the pH agent, isotonic agent. The mixture is then homogenized. Preferably, such fat emulsions contain an electric charge adjusting agent, such as acidic phospholipids, fatty acids, bilic acids, and salts thereof. Acidic phospholipids include phosphatidylserine, phosphatidylglycerol, phosphatidylinositol, and phosphatidic acid. Bilic acids include deoxycholic acid, and taurocholic acid. The preparation of such pharmaceutical compositions is described in U.S. Pat. No. 5,733,877.

The pharmaceutical compositions containing the active ingredients of the present invention as described herein above are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the medical arts. The amount must be effective to achieve improvement including but not limited to improved survival rate or more rapid recovery, or improvement or elimination of symptoms and other indicators as are selected as appropriate measures by those skilled in the medical arts. The pharmaceutical compositions can be combinations of the active ingredients but will include at least one active ingredient.

The doses can be single doses or multiple doses over a period of several days. The treatment generally has a length proportional to the length of the disease process and drug effectiveness and the patient species being treated.

In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantity to be administered will vary for the patient being treated and will vary from about 100 ng/kg of body weight to 100 mg/kg of body weight per day and preferably will be from 10 μg/kg to 10 mg/kg per day.

XVI. Knock-Out or Transgenic Animals

Transgenic Mice. The introduction of gene constructs into the genome of mice (transgenic mice) is a well-established procedure. Transgenic mice provide the opportunity to examine the phenotypic outcome of over-expression or ectopic expression of genes (gain-of-function experiments). Specific phenotypes obtained after such expression is a very strong predictor of gene function. Many human genes have been expressed in transgenic mice and in most cases they function appropriately. Thus, for the purpose of examining gain-of-function, human genes can be used. Specific plasmid vector constructs are available. They carry any of a variety of promoters that allow expression of the gene in specific tissues. For example, promoters that are brain specific are available, liver specific promoters, vascular-endothelial cell specific promoters, bone specific promoters, cardiac muscle specific promoters and many more. While mice are specifically discussed herein as the transgenic animal, those of ordinary skill in the art well understand that any other eukaryotic animal can be used in the same way as described for mice to make a corresponding transgenic animal. Transgenic mice overexpressing the 2-2-83 using the β-actin promoter and the Tet-inducible promoter gene have now been made using the known techniques and have been used in further experimentation relating to this invention; see Example 18. Using the β-actin promoter, two lines express the gene on the RNA level mainly in muscle, heart and brain. Five strains of transgenic mice have been established using the Tet-inducible promoter.

Knockout Mice. Loss-of-function experiments in mice are mostly done by the technique of gene knockout. The technology is well established. It requires the use of mouse genes for the purpose of generating knockout of the specific gene in embryonic stem (ES) cells that are then incorporated into the mouse germ-line cells from which mice carrying the gene knockout are generated. From a human gene there are several ways to recover the homologous mouse gene. One way is to use the human gene to probe mouse genomic libraries of lambda phages, cosmids or BACs. Positive clones are examined and sequenced to verify the identity of the mouse gene. Another way is to mine the mouse EST database to find the matching mouse sequences. This can be the basis for generating primer-pairs or specific mouse probes that allow an efficient screen of the mouse genomic libraries mentioned above by PCR or by hybridization. For the vast majority of genes the mouse homologue of the human gene retains the same biological function. The loss-of-function experiments in mice indicate the consequences of absence of expression of the gene on the phenotype of the mouse and the information obtained is applicable to the function of the gene in humans. On many occasions a specific phenotype observed in knockout mice was similar to a specific human inherited disease and the gene was then proved to be involved and mutated in the human disease. While mice are specifically discussed herein as the knockout animal, those of ordinary skill in the art well understand that any other eukaryotic animal can be used in the same way as described for mice to make a corresponding knockout animal. Knock-out mice lacking expression of the 2-2-83 gene have now been made using these known techniques and are being used in further experimentation relating to the present invention.

The transgenics and knock-outs of the present invention are constructed using standard methods known in the art and as set forth in U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke et al (1991), Capecchi (1989), Davies et al (1992), Dickinson et al (1993), Duff et al (1995), Huxley et al (1991), Jakobovits et al (1993), Lamb et al (1993), Pearson et al (1993), Rothstein (1991), Schedl et al (1993), Strauss et al (1993). Further, patent applications WO 94/23049, WO 93/14200, WO 94/06908, WO 94/28123 also provide information on the production of transgenic and/or knock-out mammals.

More specifically, any techniques known in the art can be used to introduce the transgene expressibly into animals to produce the parental lines of animals. Such techniques include, but are not limited to, pronuclear microinjection (U.S. Pat. No. 4,873,191); retrovirus mediated gene transfer into germ lines (Van der Putten et al, 1985); gene targeting in embryonic stem cells (Thompson et al, 1989; Mansour, 1990 and U.S. Pat. No. 5,614,396); electroporation of embryos (Lo, 1983); and sperm-mediated gene transfer (Lavitrano et al, 1989). For a review of such techniques see Gordon (1989).

Further, one parent strain instead of carrying a direct human transgene can have the homologous endogenous gene modified by gene targeting such that it approximates the transgene. That is, the endogenous gene has been “humanized” and/or mutated (Reaume et al, 1996). It should be noted that if the animal and human sequence are essentially homologous a “humanized” gene is not required. The transgenic parent can also carry an over expressed sequence, either the non-mutant or a mutant sequence and humanized or not as required. The term transgene is therefore used to refer to all these possibilities.

Additionally, cells can be isolated from the offspring which carry a transgene from each transgenic parent and that are used to establish primary cell cultures or cell lines as is known in the art.

Where appropriate, a parent strain will be homozygous for the transgene. Additionally, where appropriate, the endogenous non-transgene in the genome that is homologous to the transgene will be non-expressive. By non-expressive is meant that the endogenous gene will not be expressed and that this non-expression is heritable in the offspring. For example, the endogenous homologous gene could be “knocked-out” by methods known in the art. Alternatively, the parental strain that receives one of the transgenes could carry a mutation at the endogenous homologous gene rendering it non-expressed.

XVII. EXAMPLES

Materials and Methods

Most of the techniques used in molecular biology are widely practiced in the art, and most practitioners are familiar with the standard resource materials which describe specific conditions and procedures. However, for convenience, the following paragraphs can serve as a guideline.

General methods in molecular biology: Standard molecular biology techniques known in the art and not specifically described were generally followed as in Sambrook et al (1989) and in Ausubel et al (1989), particularly for the Northern Analysis and in situ analysis, and in Perbal (1988), and in Watson et al Polymerase chain reaction (PCR) was carried out generally as in PCR Protocols: A Guide To Methods And Applications, Academic Press, San Diego, Calif. (1990).

Reactions and manipulations involving other nucleic acid techniques, unless stated otherwise, were performed as generally described in Sambrook et al (1989) and methodology as set forth in U.S. Pat. Nos. 4,666,828; 4,683,202; 4,801,531; 5,192,659 and 5,272,057 and incorporated herein by reference.

Microarray Hybridization Analysis

Preparation of Custom Hypoxia-Specific Microarrays

The cell system for gene discovery consisted of the rat glioma cell line C6. The cells were exposed to hypoxia for 4 or 16 hours and the pattern of gene expression was compared to cells grown under normal conditions. DNA microarrays were prepared from clones of subtracted cDNA libraries enriched for sequences differentially regulated by hypoxia.

Subtracted libraries were made from the RNA populations extracted from C6 cells cultured in the following conditions:
1. 16 hours hypoxia vs. normoxia (enrichment for genes up-regulated after 16 hours hypoxia).
2. Normal vs. 16 hours hypoxia (enrichment for genes down-regulated after 16 hours of hypoxia).
3. 4 hours hypoxia vs. normal (enrichment for genes up-regulated after 4 hours of hypoxia).

Three enriched libraries from the three groups above were made by the SSH method using the “PCR select cDNA subtraction kit” from Clontech. From library 1, 1,000 colonies were grown and the plasmids prepared in 96 well format. From libraries 2 and 3, 500 colonies were processed from each. Thus, a total of 2,000 individual plasmids were prepared and used for the fabrication of a Gene Expression Microarray (GEM). For this, the inserts of each plasmid were amplified by PCR and robotically fabricated on the glass.

Preparation of Probes for Microarray Hybridization

Isolated messenger RNA was labeled with fluorescent dNTP's using a reverse transcription reaction to generate labeled cDNA probes. mRNA is extracted from C6 cells cultured in normoxia conditions and labeled with Cy3-dCTP (Amersham) from C6 cells cultured under hypoxia conditions (either 4 or 16 hours) and labeled with Cy5-dCTP (Amersham). Two differently labeled cDNA probes were then mixed and hybridized onto microarrays (Schena et al, 1996). Following hybridization the microarrays were scanned using a laser scanner and the amount of fluorescence of each of the fluorescence dyes was measured for each cDNA clone on the microarray giving an indication of the level of mRNA in each of the original mRNA populations being tested. Comparison of the fluorescence on each cDNA clone on the microarray between the two different fluorescent dyes is a measure for the differential expression of the indicated genes between the two experimental conditions.

The following probes were made from C6 and A172 for screening the GEM:
1. Normoxia (Cy3 labeled)+16 hours hypoxia (Cy5 labeled).
2. Normoxia (Cy3 labeled)+4 hours hypoxia (Cy5 labeled).

The detected sequences are divided into three categories: 1. Novel genes; 2. known genes not known before this publication to be hypoxia regulated; and 3. known genes known to be differentially regulated under hypoxia conditions. Gene 2-2-83 (SEQ ID NO:1) was identified as a novel gene fragment whose expression is down-regulated by hypoxia.

Utilizing microarray hybridization the sequences set forth herein were identified and cloned as being differentially expressed under hypoxia conditions (see also Braren et al, 1997).

In parallel assessment of 2-2-83 (SEQ ID NO:1) gene expression by Northern Analysis, the results where found to coincide with those of microarray hybridization analysis. As well in other experiments, the results from in situ hybridization analysis showed a high degree of correlation with the Northern Analysis and microarray analysis.

In Situ Hybridization Analysis

In situ hybridization analysis was performed to assess the 2-2-83 (SEQ ID NO:1) gene expression pattern in normal tissues and in pathological models as described herein.

Disease Models For In Situ Hybridization Analysis

Hypoxic Rat Retina: Hypoxia in retina was created by exposing of new born rat pups to hyperoxia which led to the reduction of blood supply. Upon transfer to normal oxygen conditions, relative hypoxia is formed. The hypoxic retina was excised, fixed, sliced and used for the hybridization with ³⁵S-dATP labeled riboprobes.

Solid Tumors: C6 rat glioma-derived solid tumors were obtained by subcutaneous injection of the suspension of C6 cell into nude mice. Sections of two tumor samples were used in in situ hybridization. One sample represented a solid tumor of about 4×3 mm in size. No significant morphological variations between different tumor regions were observed. However, at the tumor periphery, there was a region showing elevated expression of VEGF (indicative for hypoxia). The second sample represented a tumor of about 3×1 mm in size, containing a “core” region comprised of “white” thrombus and necrotic masses. This “core” region was surrounded by tumor cells forming the “wall” of varying thickness, from about five to fifteen cell layers. VEGF was found expressed by the closest to the core layer of tumor cells. The most distant cell layers showed no VEGF expression.

Middle Cerebral Artery Occlusion (MCAO) Stroke

Model: The model was implied in the stroke-prone spontaneously hypertensive rat strain. Occlusion was permanent and unilateral, and produced by electro-coagulation of MCA. This led to focal brain ischemia at the ipsilateral side of brain cortex leaving the contralateral side intact (control). Experimental animals were sacrificed 4 and 24 hours after the operation. Brains were removed, fixed in formalin, embedded into paraffin and coronal sections were performed for the further use in in situ hybridization with candidate genes-specific riboprobes. VEGF and PGK (phosphoglycerokinase, a glycolitic enzyme up-regulated by hypoxia) specific riboprobes were used as positive controls. At 24 hours post operation, a significant up-regulation of VEGF expression was revealed in the brain cortex in the areas adjacent to the ischemic core region. Heavily labeled (presumably glial) cells could be seen at the ipsilateral to the injury side. In addition, a strong hybridization signal was displayed by few cells at the contralateral side suggesting the stimulation of VEGF response through interhemispheric communication. More detail about the model can be found in Lipton (1999)

Cell Lines

C6— Rat glioma cell line. Culture conditions: DMEM supplemented with 10% FCS, 20 U/ml penicillin, 20 g/ml streptomycin.

BE2C—Differentiated human neuroblastoma cells are a suitable and reliable model for in vitro study of processes that occur in brain of patients suffering from acute and chronic neurodegenerative or hypoxic disorders. BE2C is a subclone of the SK—N-BE(2) human neuroblastoma cell line. Unlike the parental cell line, which grows as a mixed population of adherent and floating cells, BE2C cells are strictly adherent. The cells have a polygonal form and grow as clusters of flattened neuroblasts with numerous short cytoplasmic processes, while a few cells can also have one long neurite. The BE2C cells exhibit moderate levels of tyrosine hydroxylase and dopamine beta hydroxylase activity. They contain neurofilaments and specifically express D2-dopaminergic, alpha2-adrenergic, m2/m4-muscarinic and delta-opioid receptors.

BE2C was modified to express the retroviral ecotropic receptor. This manipulation made the BE2C cells suitable for retroviral gene delivery. BE2C cells are maintained in RPMI 1640 medium supplemented with 10% of heat-inactivated FCS, 2 mM L-glutamine, 1 mM sodium pyruvate, 20 U/ml penicillin, 20 mg/ml streptomycin and 0.5 μg/ml fungizon (Gibco BRL). For neuronal differentiation, cultures of the neuroblastoma cells are exposed to 40 mM of all-trans retinoic acid (RA). After 5-6 days, cells extend neurite processes and show neuronal-like differentiation. For infection or transfection experiments, confluent non-differentiated BE2C cultures are washed with PBS, detached with Trypsin-EDTA and subcultured to poly-L-lysine-coated plates at low density.

Differentiated and non-differentiated BE2C cells were tested for sensitivity to dopamine, L-Glutamate toxicity and hypoxia (0.5% O₂). Cells viability was measured by Neutral Red assay (Biorad). Type of cell death was determined by DAPI staining. Optimal experimental conditions were calibrated.

Pro- and Anti-apoptotic Activity Tests in Transient Transfection Assays

In order to evaluate the potential pro-apoptotic properties of gene 2-2-83, HeLa and 293 cells were transiently co-transfected with 4 mg of the 2-2-83 gene plasmid and 2 mg of GFP expressing plasmid. Twenty-four and forty-eight hours post transfection the cells were fixed with 4% formaldehyde and stained with DAPI.

The anti-apoptotic properties of the gene were examined in a similar assay by adding 1.2 mg of a pro-apoptotic expression construct (intracellular domain of Fas or RIP death-inducing domain) to the transfection.

The percentage of the apoptotic cells (among the GFP-expressing cells) in 2 independent experiments was calculated. For further analysis of the cell cycle and the apoptosis, 10⁶ cells from the transfectants were harvested, resuspended in 1 ml PBS containing 0.05% triton and 50 mg/ml propidium iodide (PI) and subjected to FACS analysis.

Stable Transfection of C6 Glioma Cells

C6 cells were stably transfected by pCDNA3 vector either empty or expressing a gene 2-2-83 using a lipofectamine procedure. After three weeks of G418 (1.5 mg/ml) selection, independent clones were isolated. The level of gene expression was measured by Northern blot. Total RNA samples (10 mg) from the G418 selected clones were separated on formaldehyde gels, transferred to nylon membrane, and hybridized with a 2-2-83-specific probe. C6 samples (3 mg of poly-A RNA) were taken from 16 hours hypoxia treated cells as a positive control.

Stable Transfection of Human BE2C Cells

Stable 2-2-83 expressing polyclonal cell populations were obtained by either retroviral transduction with pBABE-Puro-2-2-83 retroviral vectors into BE2C-ecotropic viral receptor expressing cells or by transfection of BE2C cells with pCDNA3-2-2-83 (Fugene 6 reagent—Boehringer). The corresponding empty vector served as control in both cases. High titer virus for infection was produced by ecotropic packaging cell line transfected with expression constructs by Ca/phosphate technique.

After either puromycin (pBabe) or G418 (pcDNA3) treatment, stable transfectants were selected as a batch (no single clones were selected). Total RNA was isolated from the cells for Northern blot analysis confirming the expression of 2-2-83.

Measuring the Sensitivity of 2-2-83 Expressing C6 Clones to Hypoxia

For the assay, the cells were plated at low density onto 96-well plates (5000 cells/well). 24 hours later, the cells were exposed to hypoxic conditions (0.4% O₂) for 3 days. Viable cells' density was estimated calorimetrically by neutral red assay. Note that there was a strong dependence between response to hypoxia and the density of cells.

Endothelial Cell Proliferation Assay

Conditioned media from 2-2-83 expressing clones was examined for its ability to induce/inhibit bovine endothelial cell (BAEC) proliferation. For the proliferation assay, the cells were plated at low density onto 96-well plates (500 cells/well). 24 hours later, the culture medium is replaced with 50 ml of DMEM supplemented with 5% calf serum and 50 ml of the test sample (condition medium from C6 cell clones expressing the candidate genes). For the inhibition assay, bFGF (0.5 ng/ml) was added one hour later. The medium was replaced 72 hours later as described above. The assay was terminated after 6-8 days by fixation with 2.5% formaldehyde and the cell density was estimated colorimetrically by staining with methylene blue.

Tumorigenesis In Nude Mice

1.5×10⁶ C6 glioma cells from stably expressing 2-2-83 and pcDNA3-GFP negative control cells were injected subcutaneously into 4 weeks male nude mice (2 clones of each gene, 3 mice per group). Cell clones were injected individually and as various mixtures with control cells. Following initial evidence of tumor development, tumor diameters are measured every second day. When individual tumors reach an average diameter of 1.5 cm², the tumor is operated. The tumors are preserved in freshly prepared 10% buffered formalin fixative for histopathological examination. Tumor vascularization are monitored in tumor sections. Blood vessel endothelial cells are visualized by incubation with anti-von Willebrand factor. Amounts of apoptotic cells in tumor samples were assessed by TUNEL staining of tumor sections.

### EXAMPLE 1

### Cloning of 2-2-83 cDNA

On Northern blots comprised from C6 (rat glioma) and A172 (human glioma) mRNA extracted from cells under hypoxic and normoxic conditions, 2-2-83 was found down regulated after 16 hours of hypoxia. The 2-2-83-specific cDNA probe hybridized to a single mRNA species of ˜4.0 Kb. Both rat and human orthologs of 2-2-83 cDNA were cloned. Their nucleotide and putative amino acid sequences are shown in SEQ ID NOs:1 and 3, respectively. The rat cDNA clone is 3838 bp long and contains an open reading frame potentially coding for a protein of 516 amino acids (SEQ ID NO:2) (nucl. 24-1572). Human cDNA is 4096 bp long and also codes for a 516 amino acid protein (SEQ ID NO:4) (nucl. 39-1587).

### EXAMPLE 2

### Bioinformatic Analysis

Protein structure and domain analysis revealed that 2-2-83 has three potential transmembrane domains between amino acids 31-51, 137-157, and 209-229 (SMART). The protein was also predicted to have an uncleavable signal peptide (PSORT). Amino acids 133-234 constitute a FAD-binding domain found in several FAD-dependent oxidoreductases (PRODOM). The search of available sequence databases revealed that human 2-2-83 nucleotide sequence is almost identical to human sequence D13643 designated as KIAA0018. The putative proteins encoded by rat and human 2-2-83 genes are close homologues of proteins found in several plant species (S71189 from Arabidopsis thaliana and P93472 from pea), and from C. elegans (017397). However, the putative protein encoded by KIAA018 cDNA (Q15392) appears truncated (390 amino acids instead of 516 amino acids encoded by human 2-2-83 gene). This is due to a frameshift mutation within the KIAA0018 nucleotide sequence which resulted in a deletion of C residue between the positions 1166-1167. The overall structure of 2-2-83 protein from different species is similar. However, the second and the third putative transmembrane domains were detected only in mammalian species (probably due to the substitution of Thr/Ser and Gly residues found in mammalian species to Asn residues in non-mammals within the second putative TM domain, and substitution of Cys to Gln within the third putative TM domain). The putative non-cleavable signal peptide also failed to be detected within the non-mammalian species. The FAD-binding domain is conserved through the evolution of 2-2-83 protein homologues (Mushegian et al, 1995).

### EXAMPLE 3

### Expression Pattern of 2-2-83 Gene in Normal Mouse Embryonal Development

Expression pattern of gene 2-2-83 in embryogenesis was studied by in situ hybridization on parasagittal sections of mouse embryos at days 12.5, 14.5 and 16.5 postconception (dpc). In most of 2-2-83 expressing cells, intensity of hybridization signal varied from weak to moderate. Only embryonic liver and sebaceous glands can be regarded as sites of strong 2-2-83 gene expression.

Central Nervous System

The hybridization signal is widely spread throughout the mouse embryo central nervous system. The strongest neural expression was found at 12.5 and 14.5 dpc stages in the ependymal layer of developing spinal cord and in brain (especially at the ventral side of brain ventricles). By the 16.5 dpc stage, the 2-2-83 expression disappears from the ependymal lining of central canal of the spinal cord as well as from the lateral and the fourth brain ventricles. However, the expression signal is still found in the third ventricle. Another prominent CNS region of 2-2-83 expression is the mantle layer (gives rise to the gray matter) of the spinal cord where hybridization signal could be seen at 12.5 and 14.5 dpc stages. This signal is preserved in some (but not all) neuroblasts of the ventral horn also at 16.5 dpc stage. A weak hybridization signal can be observed in developing brain cortex and in olfactory lobes. Neuroblasts of some of medulla oblongata and of hypothalamus nuclei (unidentified) display a weak hybridization signal at 12.5 and 14.5 dpc stages. These nuclei were absent from the available sections of 16.5 dpc embryos.

Peripheral Nervous System

The peripheral nervous system (spinal ganglia and brain ganglia) as well as the autonomous nervous system (sympathetic ganglia) are 2-2-83-positive at all studied stages.

Non Neural Ectoderm Derivatives

Teeth. Expression of 2-2-83 could be detected in teeth primordia at all stages studied. At 12.5 dpc, the hybridization signal was also evident in dental lamina, an ectodermal invagination that manifests the earliest stage of tooth formation. At 14.5 and 16.5 dpc, the signal could be seen in ameloblasts, cells destined to produce enamel.

Skin. The 2-2-83 expression in skin could not be detected before the 16.5 dpc stage when a weak signal appeared in the suprabasal cells of epidermis. Interestingly, this signal could be seen only at the ventral side of the body. At 16.5 dpc, a strong hybridization signal appeared also in the sebaceous glands in the association with developing vibrissae. Simultaneously, a weak hybridization signal also appeared in the external root sheath of vibrissae. Cells of the same type also displayed a weak expression in the hair roots of adult skin.

Heart and Vascular System. A weak hybridization signal was detectable in cardiomyocytes at 12.5 and 16.5 dpc. By 16.5 dpc, this signal had disappeared.

Urogenital System. Kidneys and adrenals are present on sections of 12.5 and 16.5 dpc embryos and are absent from the 14.5 dpc sections due to a cutting plane. At both available stages, a weak hybridization signal is seen in the tubular structures of kidneys and in adrenals. Seminiferous tubules of developing testes are 2-2-83-positive on 14.5 and 16.5 dpc sections. Unlike the adults' testes, the expression 2-2-83 pattern in embryo testes appeared uniform.

Skeletal System. The 2-2-83 gene displays a transient expression pattern in developing skeleton at 12.5 and 14.5 dpc. At 12.5 dpc stage, the hybridization signal is prominent in vertebrae primordia where it concentrates over the condensed portion of sclerotome. The signal in chondrocranium (e.g., in primordium of basioccipital bone) is weak and it can be seen in the innermost (i.e., most differentiated) cartilage. These cartilage cells show expression throughout the cartilaginous elements of skeleton also at 14.5 dpc. By 16.5 dpc, the hybridization signal disappears from the skeletal system.

Primitive Gut Derivatives. At 12.5 dpc, a weak hybridization signal can be seen in epithelial lining of all primitive gut derivatives present on studied embryo sections: esophagus, trachea, lungs, the pancreatic primordium and the midgut. The 2-2-83 expression levels in these structures appear to gradually decline at the later developmental stages. Thus, at 14.5 dpc, the hybridization signal is already undetectable in esophagus and trachea. By 16.5 dpc, the signal disappears also from lungs and pancreas. The thymus primordium is present both on 14.5 and 16.5 dpc sections. However, the 2-2-83 expression in thymus is detectable only at 14.5 dpc. Thyroid gland is present only on 16.5 dpc sections when the hybridization signal concentrates in the peripheral part of primordium. As mentioned above, the 2-2-83 hybridization signal in the liver is very prominent at all stages studied, and it is displayed by the liver parenchymal cells and not by the hematopoietic cells.

### EXAMPLE 4

### Expression pattern of Gene 2-2-83 in Normal Adult Rat Tissues

Expression of 2-2-83 was assessed by in situ hybridization to paraffin sections containing multiple adult rat tissues and was found in several types of cells.

Brain. In rat brain, in situ hybridization on sagittal and coronal sections reveals wide expression of this gene throughout the brain structures. Microscopic study shows that the hybridization signal concentrates over at least two cell types, neurons and oligodendrocytes. The intensity of hybridization signal and number of expressing cells vary between brain structures and even between cells within the same structure. In general, cells showing the strongest hybridization signal are concentrated in the posterior parts of brain: pons and medulla oblongata. However, single cells displaying very strong hybridization signal can be seen also in other brain regions, e.g., in midbrain (see below). Both white matter and gray matter within pons and medulla oblongata contain cells showing intensive hybridization signal. Heavily labeled neurons mark the nuclei of reticular formation in the gray matter. Strong expression in oligodendrocytes delineases the fibers of pyramidal tract. Single strong expressing trophic oligodendrocytes are scattered throughout white and gray matter in all brain structures.

Distinct layers of cerebellum show different hybridization patterns. No signal was detected in the molecular layer with the exception of few scattered strongly labeled (presumably neuronal) cells. Most of the Purkinje cells show hybridization signal of moderate intensity. Most of oligodendrocytes in the white matter of cerebellum are 2-2-83-negative, but single oligodendrocytes do display very high expression levels. The same irregular pattern of expression can be also observed throughout the oligodendrocytes within the cerebellar nuclei. Most of the neurons in these nuclei show moderate hybridization signal.

Significant variation in the intensity of hybridization signal is observed throughout the midbrain region. Most of neurons show weak to moderate signal while single neurons display very strong expression. These strongly expressing neurons are very prominent in periaqueductal gray matter and in the red nucleus.

Neurons of the cerebral cortex also display a variable hybridization signal intensity, and expression appears to be stronger in the deeper cell layers than in the outer ones. The maximal expression in neurons is observed in the most anterior (orbital) cortex region. Like in other areas of gray matter, here too a very strong hybridization signal can be detected in single trophic oligodendrocytes.

In hippocampal neurons, the highest intensity of 2-2-83 expression is in the CA3 nucleus, while expression in more posterior fields and in the dentate gyrus appears lower.

The pattern of 2-2-83 expression in the forebrain and midbrain regions of thalamus and hypothalamus is similar, and neurons of practically all nuclei display different hybridization signal intensity varying from weak to moderate.

The region of the lowest 2-2-83 expression in brain is presented by striatum.

Skin. The strongest hybridization signal was observed in cells within the basal layer of sebaceous glands. Basal cells are actually the stem cells which proliferate and give rise to terminally differentiated cells that fill the inner space of the gland. Terminally differentiated cells accumulate lipids within their cytoplasm and undergo apoptotic death that results in release of the fatty secrete.

Viscera. Weak expression of 2-2-83 was detected in the upper layers of urothelium and in surface epithelium of fundic stomach. Much stronger signal was observed in piloric surface epithelium.

Reproductive System. Expression of 2-2-83 was detected in rat testes: in basal cells (apparently spermatogonia) of some seminiferous tubules, probably, because of differential regulation at distinct stages of spermatogenesis.

2-2-83 is expressed in ovaries. The most prominent feature of its expression at this site is the close resemblance of the VEGF expression pattern in corpus luteum (CL): very strong hybridization signal in granulosa cells of postovulatory follicles undergoing luteinization and vascularization, and in young CL. In mature CL expression of 2-2-83 as well as of VEGF is less prominent. Another type of cells showing the similar pattern of expression of both genes are theca cells of secondary follicles. Derivatives of theca cells, lutein cells of interstitial glands, display a consistent hybridization signal with 2-2-83 while they are in general VEGF-negative.

### EXAMPLE 5

### Expression Pattern of 2-2-83 in Disease Models

Hypoxic Rat Retina. Hypoxia in retina was created by exposing of new born rat pups to hypoxia which led to the reduction of blood supply (Alon et al, 1995). Upon transfer to normal oxygen conditions, relative hypoxia is formed. The hypoxic retina was excised, fixed, sliced and used for the hybridization with ³⁵S-dATP labeled 2-2-83 specific antisense riboprobe. 2-2-83 RNA levels were found down-regulated in response to hypoxia.

Solid Tumors. C6 rat glioma-derived solid tumors were obtained by subcutaneous injection of the suspension of C6 cell into nude mice. Sections of two tumor samples were used in in situ hybridization. One sample represented a solid tumor of about 4×3 mm in size. No significant morphological variations between different tumor regions were observed. However, at the tumor periphery, there was a region showing elevated expression of VEGF (indicative for hypoxia). The second sample represented a tumor of about 3×1 mm in size, containing a “core” region comprised of “white” trombous and necrotic masses. This “core” region was surrounded by tumor cells forming the “wall” of varying thickness, from about five to fifteen cell layers. VEGF was found to be expressed by the closest to the core layer of tumor cells. The most distant cell layers showed no VEGF expression. Gene 2-2-83 displayed a uniform expression pattern in the second, necrotic, tumor sample. In the first sample, hybridization signal concentrated mainly at the tumor periphery but was notably absent from the VEGF-positive cells. Therefore, in C6 tumor, 2-2-83 also appeared as down-regulated by hypoxia.

In Situ Hybridization Study of Expression of the 2-2-83 Gene in Artificial Stroke Model

The model was implied in the stroke-prone spontaneously hypertensive rat strain. Occlusion was permanent, unilateral, by electrocoagulation of MCA. This led to focal brain ischemia (stroke) at the ipsilateral side of brain cortex leaving the contralateral side intact (control). Experimental animals were sacrificed 4 hours after the operation. Brains were removed, fixed in formalin, embedded into paraffin and coronal sections were performed to be used in in situ hybridization with 2-2-83-specific and PGK (phosphoglycerokinase, glycolitic enzyme, up-regulated by hypoxia) specific riboprobes.

Radioactive in situ hybridization was performed essentially according to the published protocol of Faerman et al (1997).

The A probe specific for the 2-2-83 gene was hybridized to coronal sections of SHR rat brains (brains from spontaneously hypertensive rats), which had been subjected to permanent right middle cerebral artery occlusion (MCAO) and fixed at different time points after the operation. A probe specific for the early response gene c-fos was used as the control of successful occlusion. Results of in situ hybridization studies regarding the intensity of hybridization signal in right (ipsilateral) vs. left (contralateral) hemispheres are summarized in Table 1.

TABLE 1
Relative Intensity of Hybridization Signals in Right
(R) and Left (L) Hemispheres

| Rat # | Time after operation | | c-fos | 2-2-83 |
|---|---|---|---|---|
| 6 | 30 | min | R > L | R = L |
| 13 | 30 | min | R > L | R = L |
| 7 | 1 | hr | R > L | R > L |
| 15 | 1 | hr | R > L | R = L |
| 9 | 2 | hrs | R > L | R > L |
| 16 | 12 | hrs | — | R > L |
| 10 | 24 | hrs | — | R > L |
| 8 | 24 | hrs | — | R > L |
| 5 | 24 | hrs | — | R > L |
| 1 | 48 | hrs | — | R = L |
| 2 | 48 | hrs | — | R = L |
| 11 | 72 | hrs | — | R = L |
| 12 | 72 | hrs | — | R = L |

In normal brain, 2-2-83 is strongly expressed in neurons of distinct areas of adult rat brain including cerebral cortex. Results of the MCAO experiment show transient upregulation of the 2-2-83 gene expression in areas adjacent to the infarction core at 1-24 hrs of occlusion. Microscopically, this elevated expression was found to be located to neurons in all cortical layers. The up-regulation of the 2-2-83 gene suggests involvement of the gene product in brain tissue response to the ischemic injury leading to delayed cell death in periinfarct area.

Parkinson's Disease Model

Dopaminergic brain lesions were induced by a unilateral stereotaxic injection of neurotoxin 6-OH-DA into the right substantia nigra of male rats (150-200 g weight). Ten days after injection, the apomorphine sensitivity test was performed. Next day, rats that displayed a rotational behavior were sacrificed, brains were excised, substantia nigra and striatum regions were dissected, fixed in 4% paraformaldehyde, embedded into paraffin and processed for in situ hybridization. No difference in the pattern of the 2-2-83 expression was found between the right (injected) and left (control) sides of substantia nigra and striatum.

### EXAMPLE 6

### Overexpression of 2-2-83 in Transient Assays Neither Induces Apoptosis Nor Protects Cells from FAS-Induced Apoptosis

Since down-regulation of 2-2-83 expression was observed in hypoxic tissues that contain many apoptotic cells (hypoxic retina, stroke penumbra, hypoxic regions of glial tumors), the potential association of 2-2-83 expression either with intrinsic apoptotic activity or with intrinsic ability to protect from apoptosis was assessed.

For this, cDNA3-2-2-83 plasmid was transiently transfected together with pcDNA3-GFP plasmid in HeLa and 293 cells. 24 and 48 hours later the cells were fixed and stained with DAPI. No apoptotic effect was observed in the transfected cells. In order to evaluate the potential anti-apoptotic properties of the 2-2-83 protein, FAS-expressing plasmid was included into the co-transfection mixture. No effect opposing FAS-induced apoptosis was observed.

### EXAMPLE 7

### Stably Overexpressing 2-2-83 C6 Glioma and BE2C Neuroblastoma Cells Display Altered Phenotype

Cell clones expressing 2-2-83 from the pcDNA3 expression vector were obtained by transfection of C6 cells. In BE2C cells, two different polyclonal cell populations stably expressing 2-2-83 either from pcDNA3 vector or from pBABE retroviral vector were obtained.

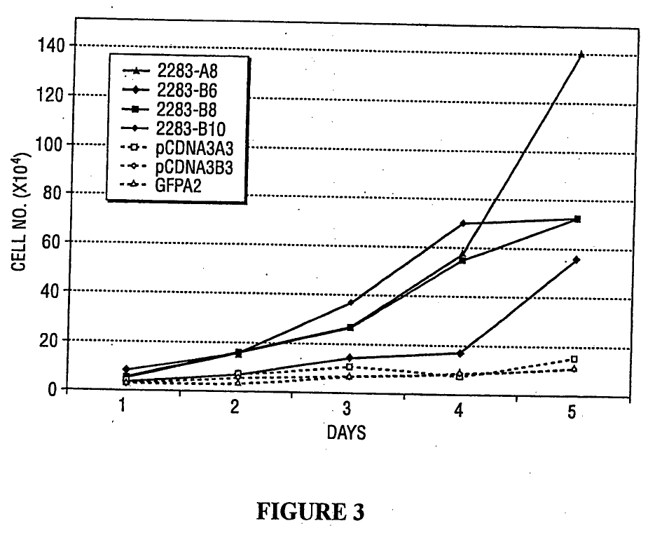

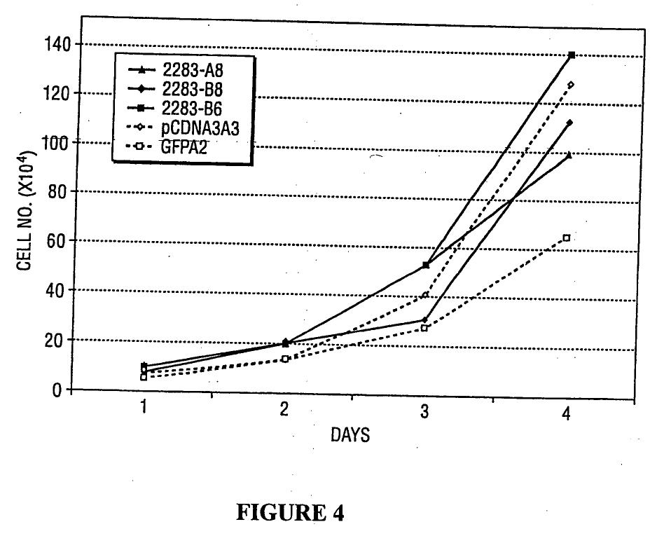

2-2-83 C6 stable cell clones have some distinct features compared to control: the cells look more flattened, tend to aggregate starting from the very low cell density, send multiple short processes and short time after transfection display higher proliferation rates than control cells reaching very high density within the aggregates (FIG. 3). They also seem to have some adhesion problems as they easily detach from plates after mechanical insults. Later on, although preserving the initial levels of exogenous 2-2-83 expression, cells slow down proliferation to the control rates (FIG. 4). 2-2-83 cells kept in culture for long periods (more than a month) proliferate slower than parental cells (see EXAMPLE 8).

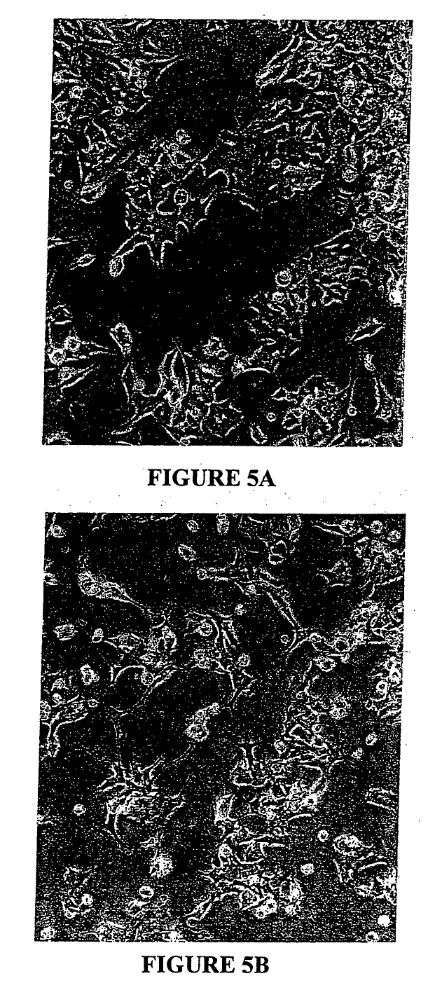

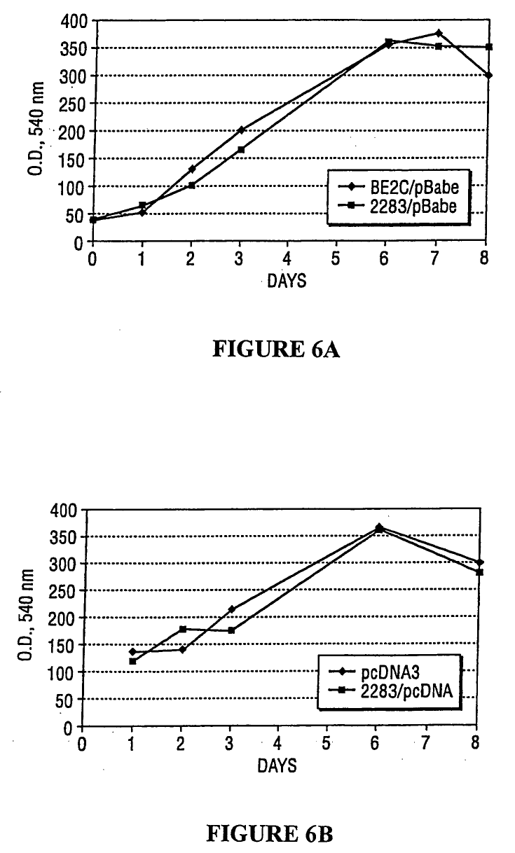

BE2C cells freshly infected with pBABE-2-2-83 send longer processes and look much more differentiated than control cells (FIGS. 5A and 5B). The proliferation rates were similar to controls (FIGS. 6A and 6B). Interestingly, FACS analysis for cell cycle distribution in BE2C cells freshly transduced with pBABE-2-2-83 revealed that it is distinct from control: relatively more cells had either less than 2n or higher than 2n DNA content, suggesting certain accumulation of apoptotic and proliferating cells in population. Since both processes compensate one another, similarity in growth curves between control and 2-2-83 expressing BE2C cells are explainable.

### EXAMPLE 8

### Control C6 Cells Being Co-Cultivated with 2-2-83 Expressing Cultures Send Longer Processes

Since plant 2-2-83 ortholog, diminuto, is involved in steroid synthesis, and steroids are molecules able to enter and to leave the cell freely, testing was done to determine if the conditioned medium of 2-2-83 expressing cells can influence the parental cells phenotype. For this, equal amounts of cells from either C6-2-2-83 cell clones (kept in culture for more than a month) or from C6-pcDNA3 cell clones (kept in culture for more than a month) were mixed with equal amount of parental C6 cells engineered to express GFP. Cells were plated and observed microscopically. First, it was immediately evident under the light microscopy, that while the mixtures of vector transfected and parental C6 cells grew as homogenous populations of small cells with typical C6 morphology, in mixed populations containing 2-2-83 expressing cells there were islands of slowly proliferating flattened cells. Analysis of cells under fluorescent microscopy revealed, that these cells were GFP-negative, hence 2-2-83 expressing. To estimate the relative amount of GFP-positive and negative cells in mixed populations, they were FACS sorted. In control cell mixtures, amount of GFP-positive (parental C6) and negative cells (vector-transfected) appeared equal, while GFP-negative 2-2-83-expressing C6 cells constituted only 20% of mixed cell population though the initial numbers of plated GFP-positive and negative cells were equal. Moreover, observation of cultured cells under fluorescent microscopy demonstrated that parental C6 cells send long processes in the direction of 2-2-83-expressing cells. This was not observed in control plates.

### EXAMPLE 9

### 2-2-83 Conditioned Medium Protects from Oxidative Stress

Experiments were done in human embryonic kidney cells (293). Cells were transfected with pCDNA3 plasmid containing the cDNA of human 2-2-83 (293-2283H) or with empty plasmid (293-control). Following neomycin selection, stable polyclonal pools were isolated. These cells were subsequently used in the following experiments.

On day 1, 293-2283 and 293 control cells were seeded on 10 cm plates (8×10⁶/plate) and grown overnight in 7 ml medium/plate (DMEM+10% fetal bovine serum+100 U/ml penicillin/streptomycin). On same day, 293-control cells were seeded on 24-well plates (1.25×10³/well) pre-coated with gelatin (0.1% w/v).

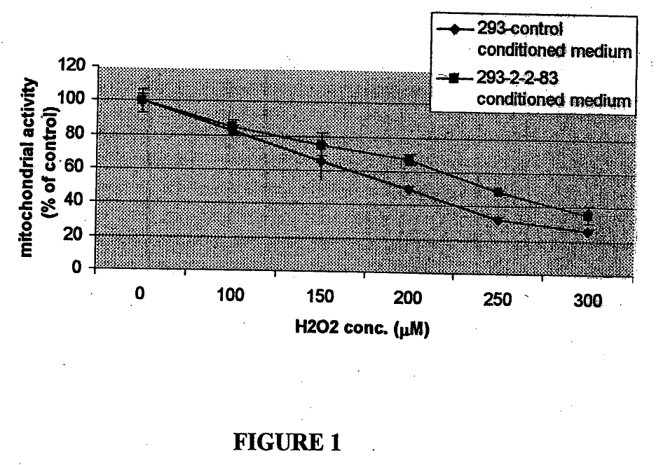

On day 2, the medium of either 293-2283 or 293-control cells in 10 cm plates (conditioned medium) was collected and H₂O₂ was added to final concentrations as shown in FIG. 1. The medium of the 24-well plates was replaced with conditioned medium. Five hours after addition of the conditioned medium, the medium was replaced by a fresh one and WST1 cell proliferation reagent was added to 5% (v/v). Aliquots were taken to 96-well plates and plates were read at 450 nm. The results are shown in FIG. 1.

On the basis of the presented experiment it is apparent that 2-2-83 produces an anti-oxidative molecule which is secreted into the medium.

### EXAMPLE 10

### C6 and BE2C₂₋₂-83 Expressing Cells Are Slightly More Resistant to Hypoxia-Induced Cell Death Than Parental Cells

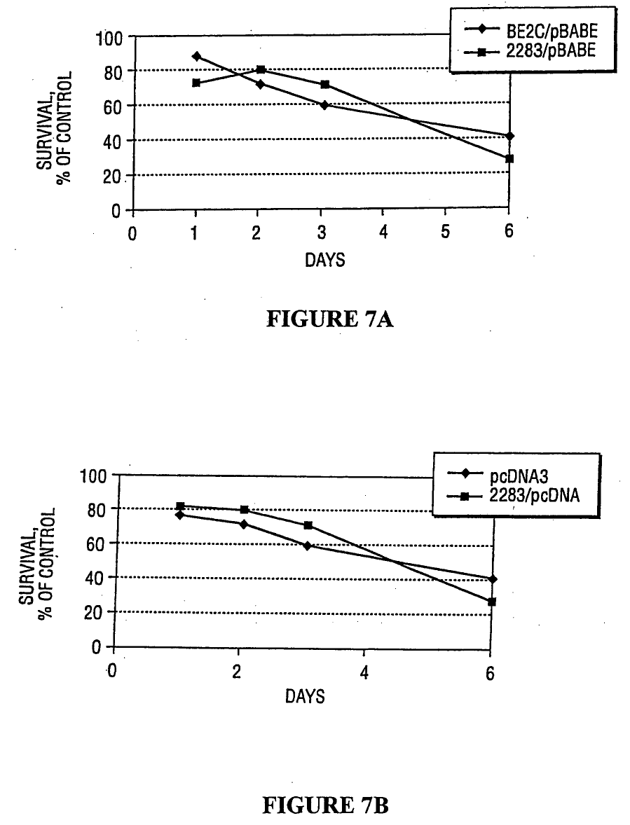

2-2-83-expressing C6 cell clones were subjected to hypoxia (0.5% oxygen) treatment for 3 days. Two polyclonal BE2C-2-2-83 cell populations were subjected to chemical hypoxia by addition of iron-chelator agent DFO (100 μM) to culture media. Both cell types that overexpress 2-2-83 appeared slightly more resistant to hypoxia-induced apoptosis than control cells (FIG. 7).

### EXAMPLE 11

### Effect of Conditioned Media from 2-2-83 Overexpressing Cells in Angiogenesis Assays

The potential involvement of 2-2-83 in regulation of angiogenic processes was studied next. In vitro angiogenic assays, including BAEC proliferation and aortic rings, did not indicate that conditioned media of C6 cells that overexpress 2-2-83, has either angiogenic or anti-angiogenic activity. Moreover, histological analysis of C6-derived tumors developed in nude mice (not shown) did not reveal any excessive angiogenesis or lack of vascularization in tumor regions that overexpress exogenous 2-2-83.

Further studies were conducted on the potential involvement of 2-2-83 gene in the processes of angiogenesis and neoangiogenesis by in situ hybridization analysis of mouse placenta—the organ where many angiogenesis-related and tissue-remodeling events occur. The strongest 2-2-83 hybridization signal on sections of 7.5 dpc decidua was seen at the periphery of the vascular zone where it concentrated over decidual (maternal) and probably some trophoblast (embryonic) cells surrounded by sinusoidal vessels. In the rest of decidua, expressing cells demarcated the boundary between decidua and uterine stromal cells.

At day 8.5 of pregnancy the pattern of decidual expression remained mainly unchanged: expressing cells were seen at the periphery of vascular zone and decidua capsularis. In addition to decidua, a weak hybridization signal could be seen in chorionic plate and visceral yolk sac. At 9.5 and 10.5 dpc stages, the number of 2-2-83 expressing cells in vascular zone significantly decreased compared to previous stages and no hybridization signal was observed in decidua capsularis. Hybridization signal was still seen in visceral yolk sac and in chorionic plate undergoing transformation into labyrinthine part of placenta. At later stages, placental expression of the 2-2-83 gene was further down-regulated, so that only single expressing cells could be identified in labyrinth and yolk sac of 13.5 and 15.5 dpc placentas (not shown).

The detected pattern of 2-2-83 expression in developing placenta again does not enable one to connect this gene to angiogenesis. Recent reports on the expression pattern of mRNAs encoding two key enzymes responsible for de novo synthesis of steroid hormones—cholesterol side chain cleavage cytochrome P450 (P450scc) and 3-beta-hydroxysteroid hydrogenase/isomerase type VI (3betaHSD VI) (Schiff et al, 1993; Arensburg et al, 1999)—demonstrated that decidual cells and giant cells of trophoblast are the sites of local progesterone synthesis during early pregnancy. It has been found that gene 2-2-83 is expressed in a rather confined subset of decidual cells and not in giant cells of trophoblast. Moreover, expression of the 2-2-83 gene was detected in chorioallantois and visceral yolk sac—structures that are not implicated in synthesis of steroid. This doubts the direct involvement of the 2-2-83 gene into placental de novo steroidogenesis either (at least into known pathways).

### EXAMPLE 12

### Cells Overexpressing 2-2-83 Display an Altered Sensitivity to 24- and 25-Hydroxycholesterol-Induced Cytotoxicity

The natural substrate of 2-2-83 in plants—24-methylcholesterol—is unknown in mammalian species. However, a structurally similar compound—24-hydroxycholesterol—is one of the major steroids in the brain. It easily passes the blood-brain barrier. However, its concentrations in brain versus serum demonstrate a peculiar age-dependent pattern, that suggests that this steroid is further metabolized in brain (Lund et al, 1999). It was proposed that 2-2-83 may be involved in metabolism of 24-hydroxycholesterol in brain, and that the observed peak of serum concentration of 24-hydroxycholesterol in 15 days old mice, may be explained by the potentially reduced expression of 2-2-83 in mouse brain at this particular age. To test this hypothesis, RNA from mouse brains at age 5, 10, 15, 20, 30, and 300 days was isolated and subjected it to Northern analysis with 2-2-83-specific probe. The initial preliminary results demonstrate a slight reduction in 2-2-83 expression levels in brains derived from mice at ages 10, 15 and 20 days (not shown).

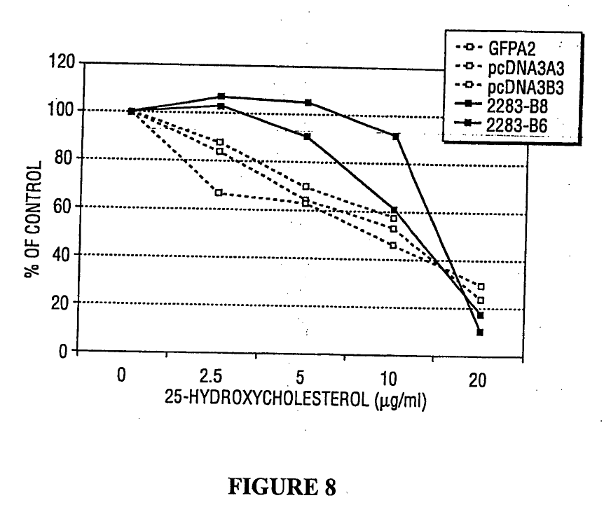

24-hydroxycholesterol and 25-hydroxycholesterol were previously shown to be toxic for neuron cells in vitro. If these steroids are 2-2-83 substrates, then the cells that overexpress 2-2-83 should be more resistant to this type of steroid-induced toxicity. Control and transfected C6 (rat glioma) cells were plated at low density onto 96-well plates (5×10³ cells/well), while control and infected BE2C non-differentiated (human neuroblastoma) cells were seeded at density 1×10⁴ cells/well. For the assay, the growth medium was replaced with 100 μl of fresh one, containing different concentrations of the hydroxycholesterols. Cell viability was estimated using the Neutral Red assay. It appeared, that overexpression of 2-2-83 in C6 cells conferred to them resistance to 25-hydroxycholesterol cytotoxicity (FIG. 8), producing no protective effect in non-differentiated BE2C cells. The observed protective effect was cell autonomous, since it was not demonstrated for control cells co-cultured together with the 2-2-83 overexpressing ones (not shown).

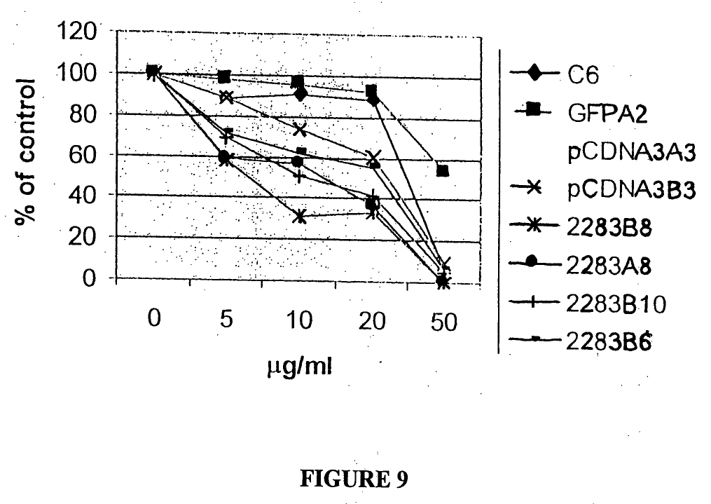

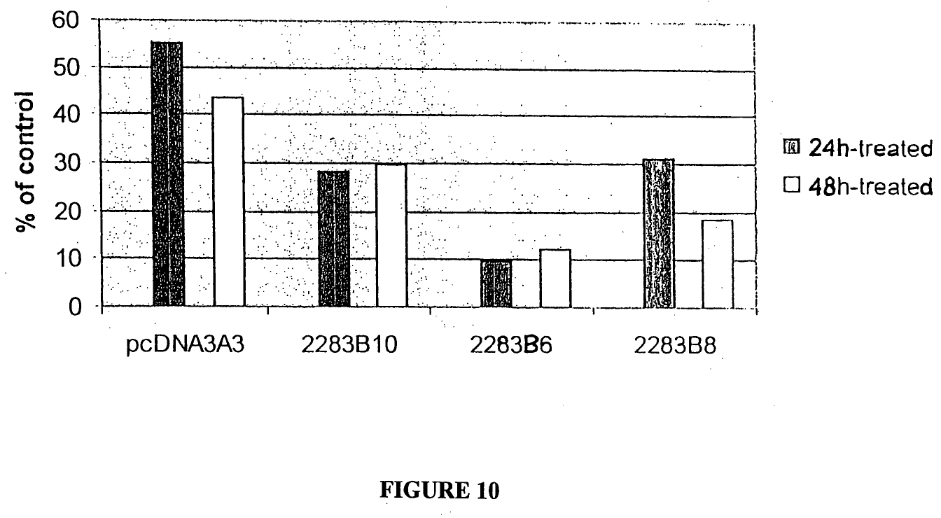

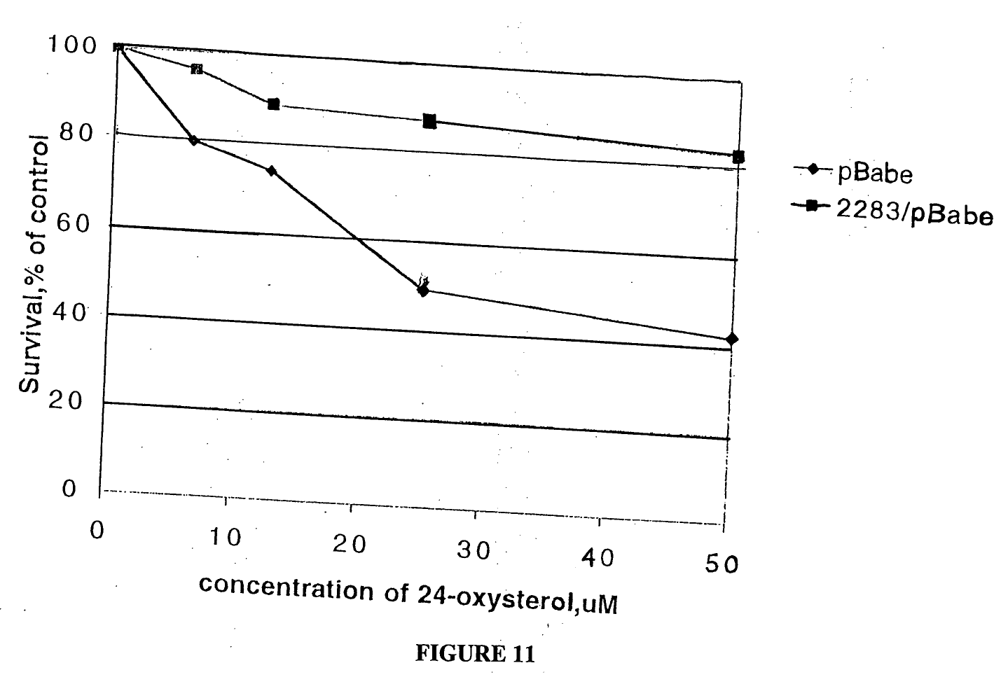

Cell response to 24-hydroxycholesterol treatment appeared different: C6 cells that overexpressed 2-2-83 became more sensitive (FIG. 9 and FIG. 10), while non-differentiated infected BE2C converted to completely resistant (FIG. 11). These results may indicate that 2-2-83 is involved in metabolism of 24- and 25-hydroxycholesterols in brain. However, it is clear that overproduction of 2-2-83 in glial and neuronal cells may have opposite consequences for their susceptibility to steroid-mediated cytotoxicity. Interestingly, overexpression of 2-2-83 either in C6 or in BE2C cells did not alter their response to hypoxia (not shown). Because of the observed induction of 2-2-83 expression in post-stroke rat brain and the potential link between 2-2-83 protein product and 24-hydroxycholesterol metabolism, it is believed that the steroid which is the ultimate product in the synthesis chain involving 2-2-83 will be useful in the treatment to ameliorate the effects of stroke.

### EXAMPLE 13

### Influence of 2-2-83 Overexpression on Tumorigenic and Metastatic Potential

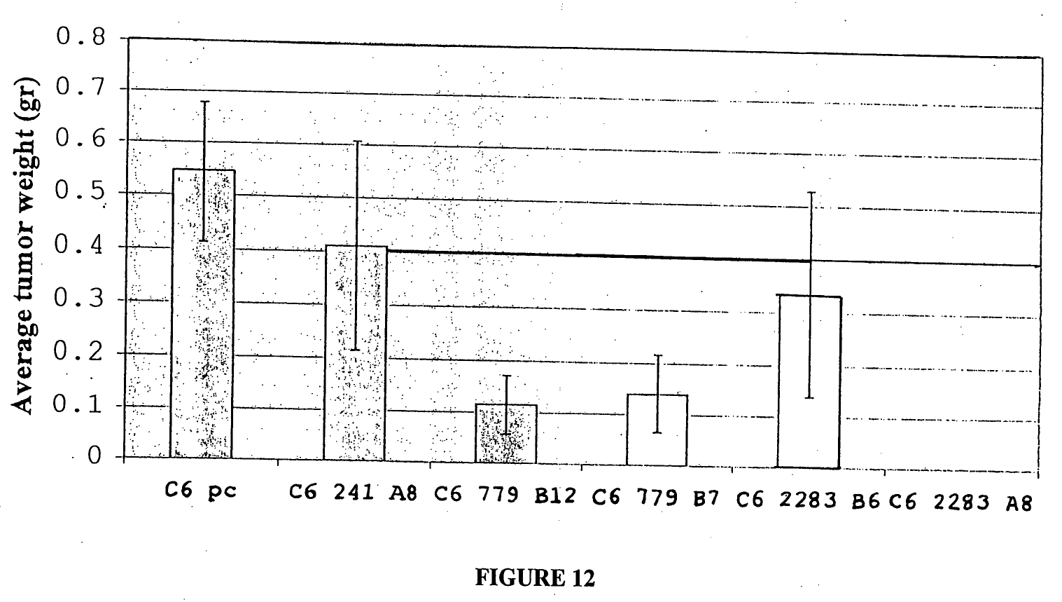

The laboratory of the present inventors next investigated whether the overexpression of 2-2-83 in tumor cells may influence their tumorigenic and metastatic potential. Assays were performed both in syngeneic and in nude mice. Control C6-pcDNA3 and two C6-2-2-83 overexpressing clones (A8 and B6) were injected subcutaneously into nude mice. Tumor growth was detectable and measurable for control injected C6-pCDNA3 cells by 11 days postinjection, while none of the C6-2-2-83 clones produced visible tumors. One of the 2-2-83 clones (A8) did not produce visible tumors even by 30 days post injection in two independent experiments. The second 2-2-83 clone (B6) gave rise to significantly smaller tumors (FIG. 12). When mixed population of C6-GFP and C6-2-2-83 cell clones were injected into nude mice in ratios 1:10 or 10:1, in both cased, tumors similar in size to control tumors were obtained. Hybridization of C₆₋₂-2-83-derived tumor sections to 2-2-83 specific probe revealed a rather weak and diffuse signal in all the tumor cells while little or no hybridization signal was observed in perinecrotic areas as was previously shown for the tumors derived from non-transfected C6 cells. In addition to endogenous 2-2-83 expression, there were also foci of cells showing a very strong 2-2-83 expression. Such foci have never been observed in tumors derived from non-transfected C6 cells and in tumors grown from C6-GFP/C6-2-2-83 mixed cell populations. Thus, this strong signal is likely to result from the expression of the transfected 2-2-83 cDNA. This suggest a very low level of survival of 2-2-83 transfectants in vivo. Interestingly, cells showing strong hybridization signal differ morphologically from the rest of tumor cells: they had small nuclei with dense chromatin and more narrow cytoplasm (not shown).

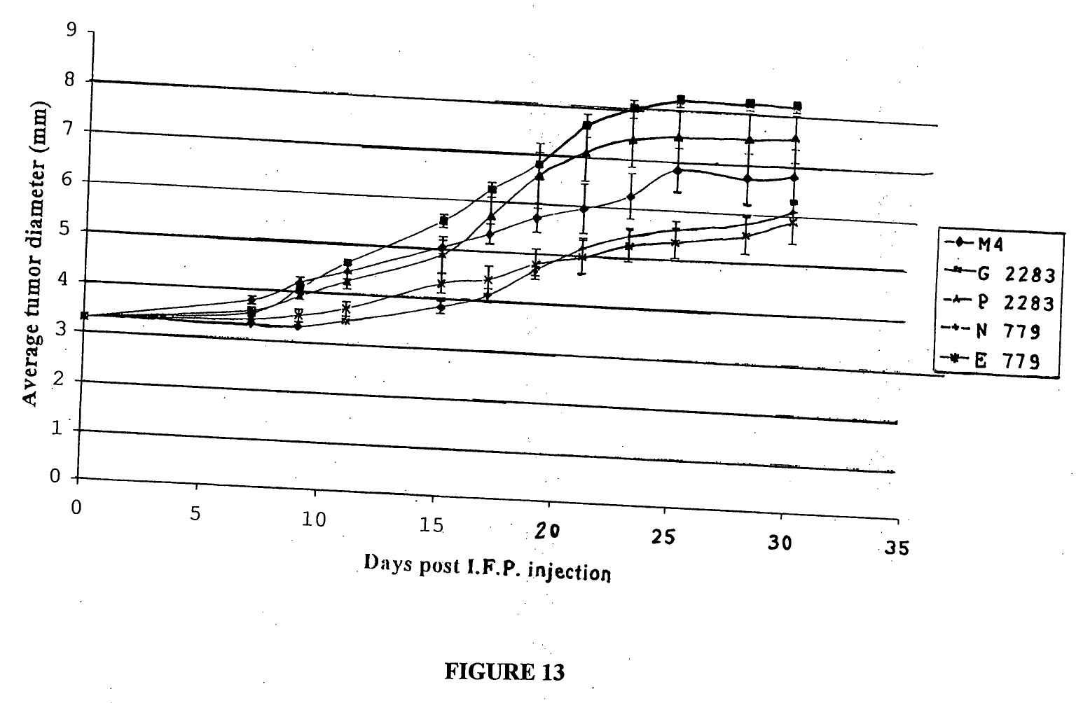

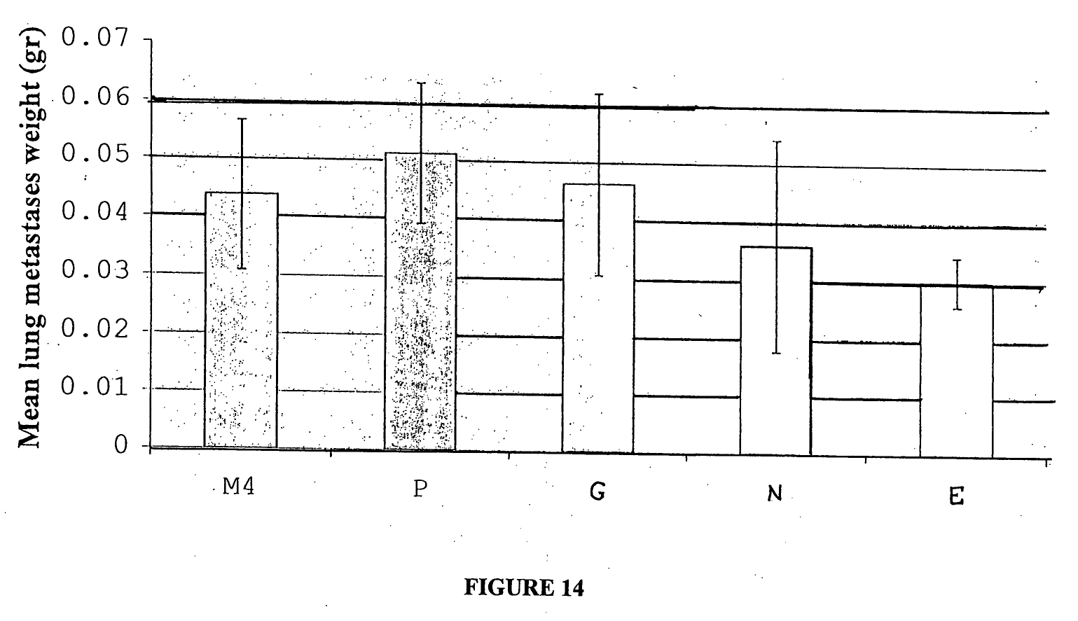

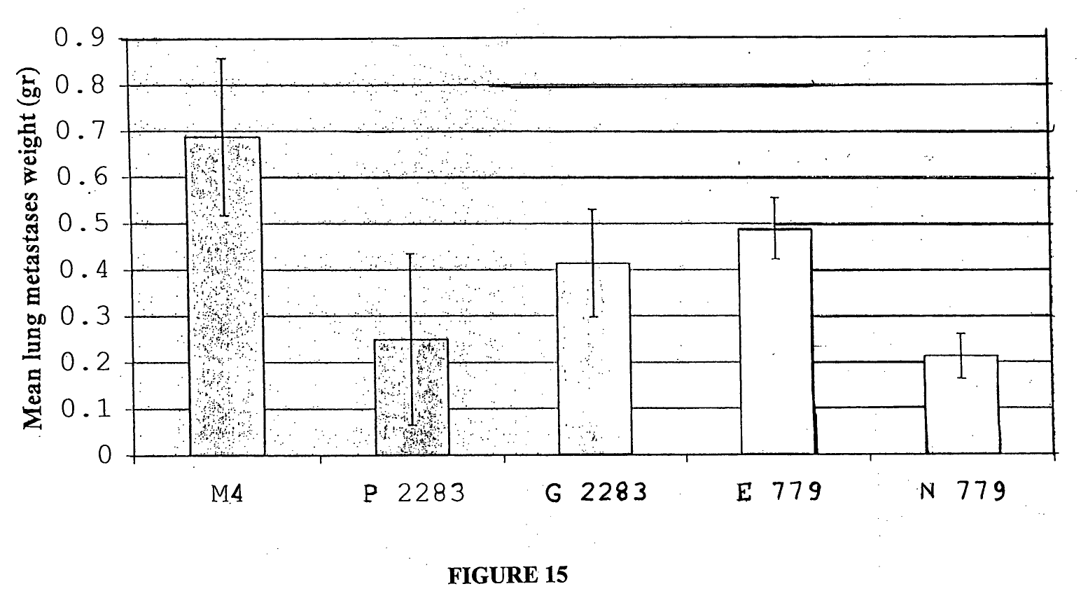

The intrafootpad tumor growth rate of M4-2-2-83 injected mice was faster compared to control M4-pCDNA3 injected mice (FIG. 13, clones P and G). Moreover, the M4-2-2-83 (G, P) clones gave rise to a higher number of lung metastases (spontaneous metastasis assay) (FIG. 14). In contrast, in the experimental metastasis assay, the same clones gave rise to significantly less lung metastases as measured by 18 days post injection (FIG. 15). Northern analysis of RNA extracted from lungs of M4-2-2-83-injected mice—spontaneous metastases (not shown) and experimental metastases revealed expression of both endogenous and exogenous 2-2-83, indicating that the metastatic process does not select against the 2-2-83 overexpressing cells. In addition, the pattern of the endogenous 2-2-83 gene expression in primary tumors versus spontaneous lung metastases derived from melanoma cells in syngeneic mice was characterized. Primary tumors of both low and highly metastatic melanomas displayed a rather weak hybridization signal in most of the cells. Cells adjacent to the areas of necrosis had little or no hybridization signal. In metastases, the expression pattern of 2-2-83 appeared different between small and large foci. In small foci, a weak hybridization signal could be usually found only in few cells while in large foci the intensity and the pattern of hybridization was similar to that in large primary tumors, potentially indicating that tumor growth selects for 2-2-83 expressing cells.

It is difficult to explain why in nude mice and in experimental lung metastasis 2-2-83 overexpression suppressed tumor formation, while in vitro, in syngeneic mice intrafootpad tumors and in spontaneous metastasis, overexpression of 2-2-83 appeared beneficial both for cell proliferation and for tumorigenicity.

### EXAMPLE 14

### Influence Of 2-2-83 Overexpression In Growth Parameters

It is known that in plants, mutations in 2-2-83 orthologous gene, diminuto, lead to dwarfism, since brassinolides (the end product of diminuto's activity) control cells' growth and proliferation. Therefore, the question of whether the overexpression of 2-2-83 may influence the growth parameters of recipient cells was investigated. The growth rate of the C6 clones stably expressing the 2-2-83 gene was followed for 5-6 days by daily cell counting. It was found that 2-2-83 expressing clones had significantly higher growth rates than the control cells (FIG. 3). However, 2-2-83 had no influence on clonogenic ability of tested HEK293 and F10.9 cells.

### EXAMPLE 15

### Point Mutant Experiment

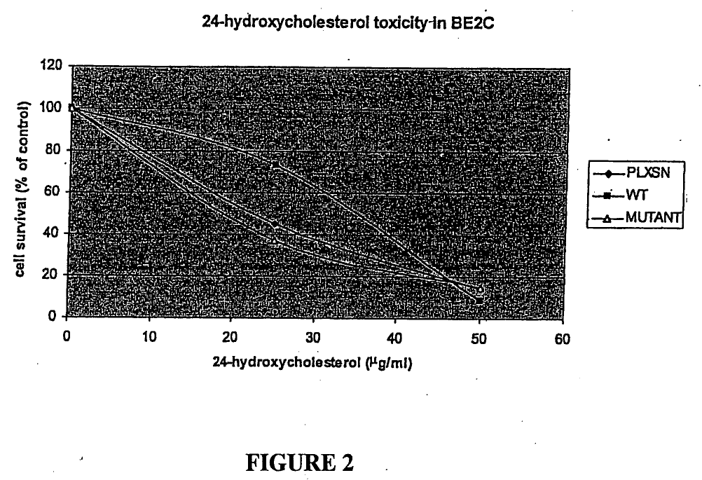

A mutant 2-2-83 molecule was created using PCR. In this mutant, glycine 181 was replaced by a valine. The mutation is in the conserved FAD binding domain and is identical to one that occurs in the diminuto gene of the dwarf 1-3 Arabidopsis plant. In the presented experiment, the results of which are shown in FIG. 2, BE2C cells infected with the mutant 2-2-83 were not protected from 24-hydroxycholesterol toxicity whereas BE2C infected with the wild type 2-2-83 were partially protected. The fact that the point mutant loses protection from 24-OH-cholesterol toxicity shows that the mutant mimics the one that naturally occurs in the plants and is known to abolish enzymatic activity of the plant diminuto. This supports the conclusion that 2-2-83 is an enzyme as is suggested by its similarity to a plant enzyme.

### EXAMPLE 16

### Ability of PC12 Clones Which Overexpress 2-2-83 To Withstand NGF Withdrawal And Ischemia

It has previously been established that BE2C cells overexpressing 2-2-83 are less sensitive to 24-hydroxycholesterol, dopamine and H₂O₂ mediated cytotoxicity, while CG cells overexpressing 2-2-83 are less sensitive to 25-hydroxycholesterol and H₂O₂-mediated cytotoxicity. To test whether 2-2-83 is able to attenuate cell death triggered by the stimuli of another type, stable PC12 clones that overexpress 2-2-83 (both constitutive and inducible expression) were established and tested for their ability to withstand NGF withdrawal and ischemia.

Materials and Methods

Maintenance of PC12 Cells. Cells were grown in DMEM medium containing glutamine, 10% horse serum (HS) and fetal bovine serum (FBS).

Generation of Stable PC12 Clones. PC12 cells were transfected with either pcDNA3 or pcDNA3-2-2-83-flag plasmid DNA using the Fugen6 reagent. 48 hours post transfection, the cells were grown in selective medium (containing G418) for 2 months until individual colonies were isolated.

Verification of 2-2-83 Expression by Immunoblotting. Individual colonies were grown and cells were harvested in Laemli sample buffer. Total protein was assayed by measuring OD at 280 nm. The whole cell extracts were resolved by SDS-PAGE and immunoblotted with polyclonal anti-2-2-83 antibody (raised in QBI).

Generation of Stable Clones of 2-2-83 in the Tet-off System. PC12-Tet-off cells (Clontech) were co-transfected with pTRE2 expressing 2-2-83 under the control of tet-repressible promoter and with pTK-hyg for antibiotic selection. Stable clones were isolated.

Verification of Inducible 2-2-83 Expression by Northern Blotting. PC12-Tet-off clones were grown in the absence or presence of 1 μg/ml tetracycline for 48 hrs. Total RNA was isolated by Easy RNA procedure and 20 μg of it were fractionated on 1% agarose gel, transferred to Nitran membrane and probed with 2-2-83-specific probe.

Differentiation of PC12. PC12 cells were seeded (20,000 cells/well) on 24-well plates pre-covered with poly-D-lysine (0.001% w/v). The cells were grown for 7 days in differentiation medium (growth medium supplemented with 2% HS, 5% FBS, and NGF (5 ng/ml, 2S subunit, Chemicon)). Only differentiated neuronal PC12 cells were used for the described toxicity models.

Toxicity Models. For NGF withdrawal experiments, the medium was replaced by differentiation medium lacking NGF and the cells were assayed 24-48 hours later. For ischemia experiments, the cells were incubated with DMEM medium lacking glucose and supplemented with 10% HS and 5% FBS. The cultures were then kept in hypoxia conditions for 16 hours.

Biochemical Assays

Lactate Dehydrogenase (LDH) Release. Medium aliquots were tested for LDH activity in comparison to activity of total cell lysates. The LDH activity was measured at OD 490 nm using the LDH kit. Cell death was estimated according to the ratio between the LDH activity in conditioned medium and in the corresponding expressed as the % of medium/total LDH activity.

Cell Proliferation Reagent WST1. WST1 reagent was added to living cells at concentration of 10% (v/v). The colored reaction is proportional to mitochondrial activity and is read at 450 nm. The color intensity is proportional to mitochondrial activity.

Results

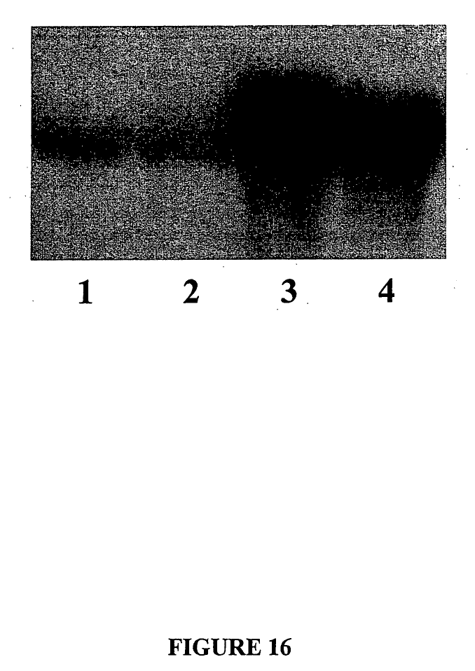

Three stable PC12 clones over expressing 2-2-83 were established (FIG. 16).

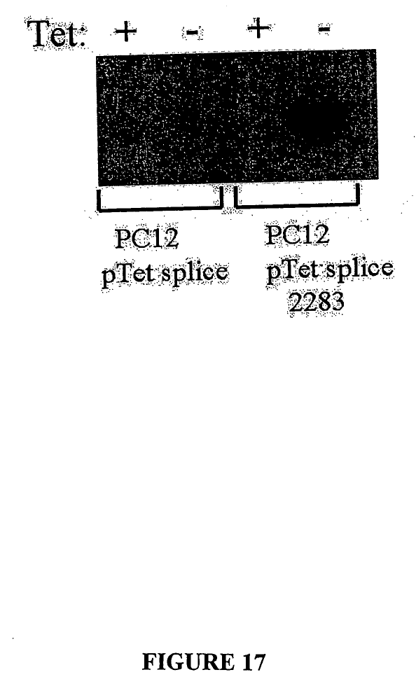

At least 3 stable PC12-Tet-off clones were found to express 2-2-83 mRNA in Tet-regulated manner (FIG. 17).

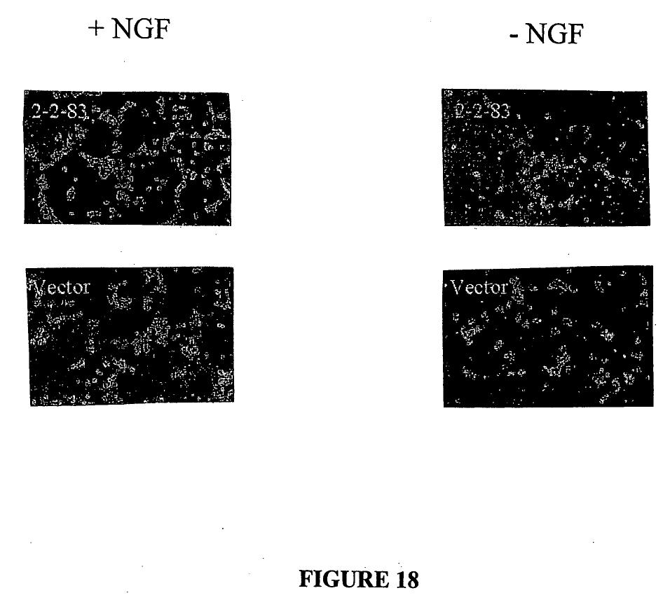

Differentiated PC12 cells overexpressing 2-2-83 were found to be partially protected from apoptosis induced by NGF withdrawal compared to control cells. They retained some of their processes (FIG. 18).

Discussion and Conclusions

The data accumulated to date on the effect of 2-2-83 expression on response of different cells to various cytotoxic (neurotoxic) stimuli (toxic steroid application, oxidative stress, ischemia, growth factor withdrawal) suggest that gene 2-2-83 affects cellular response to neurotoxic stimuli in vitro in a complex manner. In some cases it seemed to be protective (oxidative stress, toxic steroids, NGF withdrawal) whereas in another model (ischemia) it had negative contribution.

### EXAMPLE 17

### Genomic Clone of Mouse 2-2-83 Homolog

The initial gene information for 2-2-83 was the rat full-length cDNA sequence (SEQ ID NO:1). This sequence served as a basis for isolating from public gene databases the sequences of the homologous mouse cDNAs. Primer pairs were designed from the region close to the translation initiation site of the cDNAs and used to screen a mouse phage genomic library. The phage library was constructed in single clones found in 96-well plates. Plates were pooled into “pool plates”. Initial PCR was done on samples from the “pool plates”. Positive wells indicated which of the original plates contained the positive clones. PCR on the original plates was used to isolate the positive clones.

Initial sequencing was done using the primers used for PCR (above), additional sequences derived from the mouse cDNA sequence, and primers from the flanking sequence of the vector. Additional primers were designed from the sequences obtained. The partial cDNA sequence of the mouse genomic 2-2-83 homolog in SEQ ID NO:5.

### EXAMPLE 18

### Experimental Work Using Transgenic Mice Which Overexpress the 2-2-83 Gene

Materials and Methods

Animals

For both MCAO and RNA preparation, FVB/N male mice, 15-16 weeks of age and weighing 25-30 g were used.

Permanent MCAO model

Operation: Anesthesia was induced by Equithesine i.p. (3-4 ml/kg). Rectal temperature was measured and mice maintained normothermic by heating pad. The left MCA is exposed using a subtemporal approach, leaving the zygomatic arch intact. The animals are placed in lateral recumbency and a 1-cm vertical skin incision made between the left orbit and the external auditory canal. The underlying fascia is removed and the exposed temporalis muscle bluntly dissected and retracted to expose the inferior part of the temporal fossa. A small craniectomy is made using a dental drill at the junction between the medial wall and the roof of temporal fossa, approximately 0.5 mm dorsal to the foramen ovale. The dura mater is removed, and main truck of MCA is exposed proximal to the olfactory tract, and occluded by micropolar coagulation. The occluded MCA is severed to prevent recanalization. The muscle and skin were sutured by 3/0 or 4/0 Silk. Mice were housed and have access to softened food and water. Control group was operated as above except MCA was not occluded before suturing.

Staining and Preparation of Slices: Transcardial perfusion was made 24 hours after operation using TTC for 7 min. Brains were then incubated for additional 30 min at 37° C. in TTC and then incubated in formalin for 48-72 hours at room temperature. Block preparation of whole brain was done in gelatin for overnight at 37° C. Gelatin blocks were incubated in formalin for 24 hrs. and 300 μm slices were then prepared using OTS-4000. Imaging was done by Spot Digital Camera. Image analysis was done by “ImagePro-Plus” software.

RNA preparation

RNA was prepared by EZ-RNA kit (Biological Industries) essentially as described by manufacturer.

Northern Blot Analysis

Northern blot analysis was done essentially as described by Alwine et al (1977).

Summary of Results And Conclusions

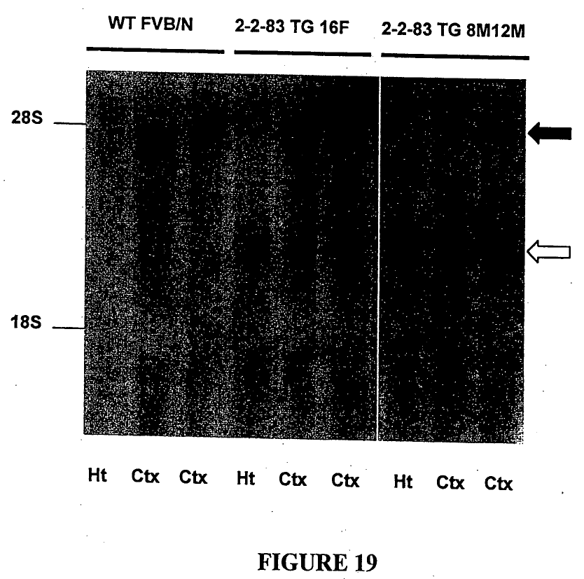

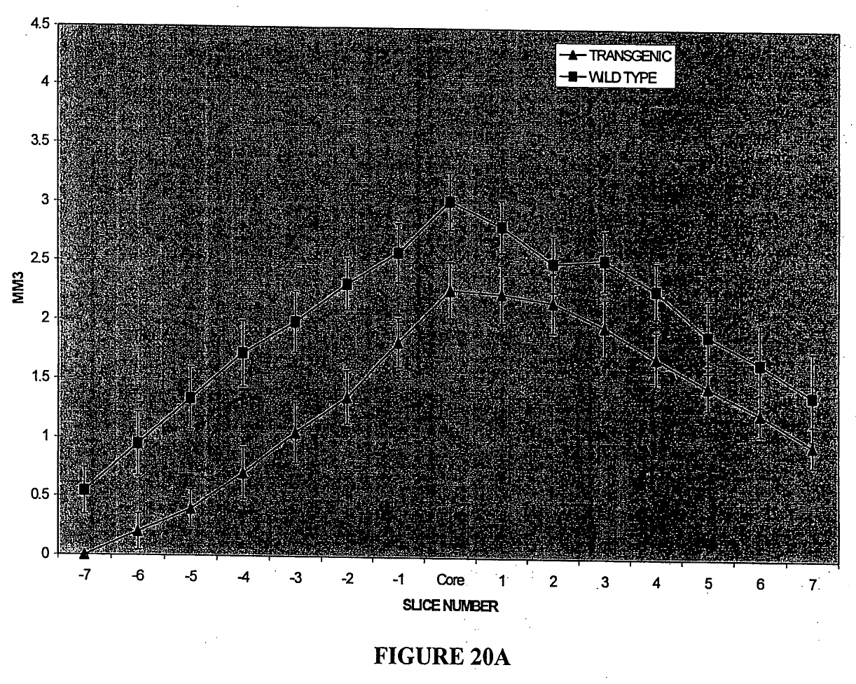

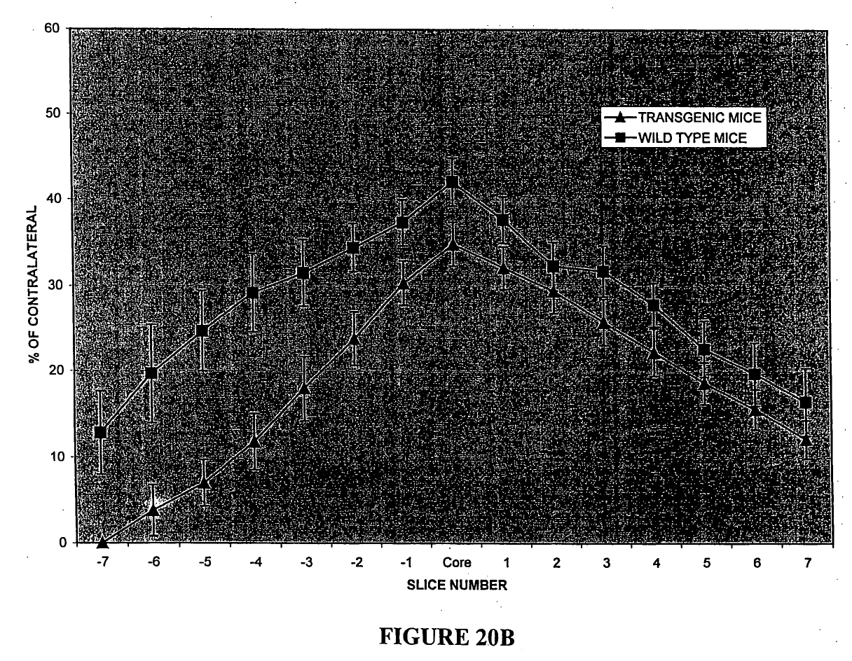

FIG. 19 shows expression of exogenous 2-2-83 mRNA in the heart and in the cortex of both TG lines. In contrast, the WT mice lack this exogenous expression. As shown in FIGS. 20A and 20B, 2-2-83 TG mice have significantly reduced infarct size as compared to their WT littermates. This indicates that 2-2-83 has a neuroprotective activity in the central nervous system and can be used for the treatment of stroke and other neurological conditions.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention. Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

### REFERENCES

Alon et al, “Vascular endothelial growth factor acts as a survival factor for newly formed retinal vessels and has implications for retinopathy of prematurity”, Nat Med 1(10):1024-1028 (1995)
Alwine et al, “Method for detection of specific RNAs in agarose gels by transfer to diazobenzyloxymethyl-paper and hybridization with DNA probes”, Proc Natl Acad Sci USA 74:5350-5354 (1977)
Arensburg et al, “Expression of steroidogenic genes in maternal and extraembryonic cells during early pregnancy in mice” Endocrinology 140(11):5220-5232 (1999)
August et al (Eds.), Gene Therapy in Advances in Pharmacology Vol. 40, Academic Press (1997)
Ausubel et al, Current Protocols in Molecular Biology, John Wiley and Sons, Baltimore, Md. (1989)
Bouck et al, “How tumors become angiogenic”, Adv Cancer Res 69:135-174 (1996)
Bradley et al, U.S. Pat. No. 5,614,396, “Methods for the genetic modification of endogenous genes in animal cells by homologous recombination”, Mar. 25, 1997
Brady et al, U.S. Pat. No. 4,609,725, “Cardiac atrial peptides”, Sep. 2, 1986
Braren et al, “Use of the EST database resource to identify and clone novel mono(ADP-ribosyl)transferase gene family members”, Adv Exp Med Bio 419:163-168 (1997).
Bunn et al, “Oxygen sensing and molecular adaptation in hypoxia”,Physiol Rev 76:839-885 (1996).
Byrne et al, U.S. Pat. No. 5,221,778, “Multiplex gene regulation”, Jun. 22, 1993
Capecchi et al, U.S. Pat. No. 5,487,992, “Cells and non-human organisms containing predetermined genomic modifications and positive-negative selection methods and vectors for making same” Jan. 30, 1996
Capecchi et al, U.S. Pat. No. 5,464,764, “Positive-negative selection methods and vectors”, Nov. 7, 1995
Carmeliet et al, “Role of HIF-lalpha in hypoxia-mediated apoptosis, cell proliferation and tumor angiogenesis”, Nature 394(6692):485-490 (1998)
Chang et al, Somatic Gene Therapy, CRC Press, Ann Arbor, Mich. (1995)
Choe et al, “The DWF4 gene in Arabidopsis encodes cytochrome P450 that mediated multiple 22-alpha-hydrohylation steps in brassinosteroid biosynthesis”, Plant Cell 10:231-243 (1998)
Clouse SD “Plant hormones: brassinosteroid in the spotlight”, Curr Biol 6:658-661 (1996)
Cordel B, U.S. Pat. No. 5,387,742, “Transgenic mice displaying the amyloid-forming pathology of Alzheimer's disease”, Feb. 7, 1995
Culver, “Site-Directed recombination for repair of mutations in the human ADA gene” (Abstract) Antisense DNA & RNA based therapeutics, February, 1998, Coronado, Calif. (1998)
Dor et al., “Ischemia-driven angiogenesis”, Trends Cardiovasc Med 7:289-294 (1997)
Duke et al, “Cell Suicide in Health and Disease”, Sci Am 275(6):80-87 (1996)
Faerman et al, “Transgenic mice: production and analysis of expression”, Methods Cell Biol 52:373-403 (1997)
Feldmann K A, “T-DNA insertion mutagenesis in Arabidopsis: Mutational spectrum”, Plant J 1:71-82 (1991).
Frossard P, U.S. Pat. No. 4,801,531, “Apo AI/CIII genomic polymorphisms predictive of atherosclerosis”, Jan. 31, 1989
Gearhart et al, WO 94/23049, “The Introduction and Expression of Large Genomic Sequences in Transgenic Animals” Oct. 13, 1994
Gilboa et al, “Transfer and expression of cloned genes using retroviral vectors”, BioTechniques 4(6):504-512 (1986)
Goldberg et al, U.S. Pat. No. 5,225,347, “Therapeutic ribozyme compositions and expression vectors”, Jul. 6, 1993
Graeber et al, “Hypoxia-mediated selection of cells with diminished apoptotic potential in solid tumours”, Nature 379(6560):88-91 (1996)
Gregory et al, U.S. Pat. No. 5,882,877, “Adenoviral vectors for gene therapy containing deletions in the adenoviral genome”, Mar. 16, 1999
Gusella J, U.S. Pat. No. 4,666,828, “Test for Huntington's disease”, May 19, 1987
Hurwitz et al, U.S. Pat. No. 5,846,546, “Preparation and use of viral vectors for mixed envelope protein immunogenic composition against human immunodeficiency viruses”, Dec. 8, 1998
Kauschmann et al, 1996
Klahre et al, “The Arabidopsis DIMINUTO/DWARF1 gene encodes a protein involved in steroid synthesis”, Plant Cell 10(10):1677-1690 (1998)
Krimpenfort et al, U.S. Pat. No. 5,175,384, “Transgenic mice depleted in mature T-cells and methods for making transgenic mice”, Dec. 29, 1992
Leder et al, U.S. Pat. No. 5,175,383, “Animal model for benign prostatic disease”, Dec. 29, 1992
Leder et al, U.S. Pat. No. 4,736,866, “Transgenic non-human mammals”, Apr. 12, 1988
Li et al, “A putative leucine-rich repeat receptor kinase involved in brassinosteroid signal transduction”, Cell 90:929-938(1997a)
Li et al, “Conservation of function between mammalian and plant steroid 5alpha-reductases”, Proc Natl Acad Sci USA 94:3554-3559 (1997b)
Li et al, “A role for brassinosteroids in light-dependent development of Arabidopsis”, Science 272:398-401 (1996)
Lipton P, “Ischemic cell death in brain neurons”, Pysiol Rev 79(4):1431-1568 (1999)
Littman et al, WO 94/06908, “Transgenic Non-Human Animals Having Targeted Lymphocyte Transduction Genes” Mar. 31, 1994
Lozano et al, “Inhibition of C-28 and C-29 physterol metabolism by N,N-dimethyldodecanamine in nematode Caenorhabditis elegans”, Lipids 20:158-166 (1996)
Lund et al, “cDNA cloning of cholesterol 24-hydroxylase, a mediator of cholesterol homeostatsis in the brain”, Proc Natl Acad Sci USA 96:7238-7243 (1999)
Marshak et al, “Strategies for Protein Purification and Characterization. A laboratory course manual, CSHL Press (1996)
McMorris TC, “Recent developments in the field of plant steroid hormones” Lipids 32:1303-1308 (1997)
Meinkoth et al, “Hybridization of nucleic acids immobilized on solid supports”, Anal Biochem 138:267-284 (1984)
Mullis K, U.S. Pat. No. 4,683,202, “Process for amplifying nucleic acid sequences”, Jul. 28, 1987
Mushegian et al, “A putative FAD-binding domain in a distinct group of oxidases including a protein involved in plant development”, Protein Sci 4:1243-1244 (1995)
Nes et al, “Lack of mammalian reduction or alkylation of 24-methylenecholesterol”, J Biol Chem 248:484-487 (1973)
Nomura et al, “Prediction of the coding sequences of unidentified human genes. I. The coding sequences of 40 new genes (KIAA0001-KIAA0040) deduced by analysis of randomly sampled cDNA clones from human immature myeloid cell line KG-1”, DNA Res 1(1):27-35 (1994)
Orsolini et al, U.S. Pat. No. 5,439,688, “Process for preparing a pharmaceutical composition”, Aug. 8, 1995
Perbal, A Practical Guide to Molecular Cloning, John Wiley & Sons, New York (1988)
Quertermous et al, U.S. Pat. No. 5,288,846, “Cell specific gene regulators” Feb. 22, 1994
Sambrook et al (Eds), Molecular Cloning: A Laboratory Manual, second edition, Cold Spring Harbor Laboratory Press, Cold Spring Harbor, (NY, 1989, 1992)
Sato et al, U.S. Pat. No. 5,733,877, “Pharmaceutical composition containing biologically active peptide or protein”, Mar. 31, 1998
Schena et al, “Parallel Human Genome Analysis: Microarray-based Expression Monitoring of 1000 genes”, Proc Natl Acad Sci USA 93(20):10614-10619 (1996)
Schiff et al, “Expression and cellular localization of uterine side-chain cleavage cytochrome P450 messenger ribonucleic acid during early pregnancy in mice”, Endocrinology 133(2):529-537 (1993)
Schwartz et al, U.S. Pat. No. 5,298,422, “Myogenic vector systems”, Mar. 29, 1994
Simons M, U.S. Pat. No. 5,192,659, “Intron sequence analysis method for detection of adjacent and remote locus alleles as haplotypes”, Mar. 9, 1993
Smulson et al, U.S. Pat. No. 5,272,057, “Method of detecting a predisposition to cancer by the use of restriction fragment length polymorphism of the gene for human poly(ADP-ribose) polymerase”, Dec. 21, 1993
Sorge J, U.S. Pat. No. 5,347,075, “Mutagenesis testing using transgenic non-human animals carrying test DNA sequences”, September 13, 1994
Szekeres et al, “Brassinosteroids rescue the deficiency of CYP90, a cytochrome P450, controlling cell elongation and deterioration in Arabidopsis” Cell 85:171-182 (1996)
Takahashi et al, “The DIMINUTO gene of Arabidopsis is involved in regulating cell elongation”, Genes Dev 9:97-107 (1995.).
Thompson et al, WO 94/28123, “Mammals Lacking Expression of CD28 Transgenic”, Dec. 8, 1994
Uda et al, U.S. Pat. No. 5,554,378, “Pharmaceutical composition and its mucosal use”, Sep. 10, 1996
Vega et al, Gene Targeting, CRC Press, Ann Arbor, Mich. (1995)
Wadsworth et al, WO 93/14200, “Transgenic Animal Models for Alzheimer's Disease”, Jul. 22, 1993
Wagner et al, U.S. Pat. No. 5,175,385, “Virus-resistant transgenic mice”, Dec. 29, 1992
Wagner et al, U.S. Pat. No. 4,873,191, “Genetic transformation of zygotes”, Oct. 10, 1989
Wang et al, “Monitoring gene expression profile changes in ovarian carcinomas using cDNA microarray”, Gene 229(1-2):101-108 (1999)
Watson et al, Recombinant DNA, Scientific American Books, New York
Weinshank et al, U.S. Pat. No. 5,360,735, “DNA encoding a human 5-HT.sub.1F receptor, vectors, and host cells”, Nov. 1, 1994
Ziao et al, Nucleic Acids Res 24:2630-2622 (1996)

1. A method for protecting cells from oxidative stress, comprising administering to an individual in need thereof an effective amount of an enhancer of transcription of a naturally-occurring gene which encodes the protein having the sequence of SEQ ID NO:4 or a protein having at least 95% identity to the sequence of SEQ ID NO:4, or by administering an enhancer of the enzymatic activity of the protein encoded by said gene.

2. A method in accordance with claim 1 for the treatment of stroke, wherein the individual in need thereof is one suffering from the effects of stroke.

3. A method in accordance with claim 1 for protecting cells from oxidative stress, comprising administering to an individual in need thereof an effective amount of an enhancer of transcription of said gene.

4. A method in accordance with claim 3 for the treatment of stroke, wherein the individual in need thereof is one suffering from the effects of stroke.

5. A method in accordance with claim 1 for protecting cells from oxidative stress, comprising administering to an individual in need thereof an effective amount of an enhancer of the enzymatic activity of the protein encoded by said gene.

6. A method in accordance with claim 5 for the treatment of stroke, wherein the individual in need thereof is one suffering from the effects of stroke.

7. An isolated polynucleotide having the sequence of
(a) SEQ ID NO:1 or SEQ ID NO:3;
(b) a naturally-occurring polynucleotide comprising a sequence of (a); or
(c) a naturally-occurring polynucleotide having at least 70% identity with a naturally-occurring polynucleotide of (b) and, in naturally-occurring neural cells, has its expression modulated when the cells are subjected to neurotoxic stress;
(d) a naturally-occurring polynucleotide capable of hybridizing under moderately stringent conditions to a naturally-occurring polynucleotide of (b) and, in naturally-occurring neural cells, has its expression modulated when the cells are subjected to neurotoxic stress;
(e) a fragment of a polynucleotide of (a), (b), (c) or (d) having at least 20 nucleotides; or
(f) a polynucleotide sequence complementary to a polynucleotide of (a), (b), (c), (d) or (e).

8. An isolated polynucleotide in accordance with claim 7, wherein said sequence of (a) is SEQ ID NO:3.

9. An isolated polynucleotide in accordance with claim 7 comprising a strand of a full-length cDNA.

10. An isolated polynucleotide in accordance with claim 9 comprising a strand of a full-length cDNA.

11. An isolated polypeptide comprising a protein encoded by a cDNA in accordance with claim 9, a variant which has an amino acid sequence having at least 70% identity to said protein and retains the biological activity thereof, or a fragment of said protein or variant which retains the biological activity thereof, or a functional derivative or salt of said protein, variant or fragment.

12. An isolated polypeptide comprising a protein encoded by a cDNA in accordance with claim 10, a variant which has an amino acid sequence having at least 70% identity to said protein and retains the biological activity thereof, or a fragment of said protein or variant which retains the biological activity thereof, or a functional derivative or salt of said protein, variant or fragment.

13. A molecule which comprises the antigen-binding portion of an antibody specific for a protein, variant or fragment in accordance with claim 11.

14. In a method for screening drugs which up-regulate or down-regulate a gene, the improvement wherein said gene is a gene which is transcribed to an RNA containing a sequence in accordance with claim 7.

15. A process for identifying a chemical compound which specifically up-regulates the 2-2-83 gene, which comprises:
contacting cells, transfected with and expressing DNA encoding the 2-2-83 gene under conditions permitting expression of the DNA, with a molecule to be screened:
determining if the molecule up-regulates the 2-2-83 gene as compared to a control; and
identifying the molecule as a potential drug if said determining procedure determines that said molecule up-regulates the 2-2-83 gene as compared to a control.

16. A method in accordance with claim 15, further including the step of producing any molecule so identified.

17. A method for alleviating or reducing damage to the central nervous system in a patient who has suffered an injury to the central nervous system, the method comprising administering to the patient a polypeptide in accordance with claim 11, in a sufficient dosage to alleviate or reduce the damage.

18. The method of claim 17 wherein said injury is an ischemic episode.

19. The method of claim 17 where an additional pharmaceutically effective compound is administered in conjunction with the pharmaceutically effective compound.

20. The method of claim 18, wherein said ischemic episode is caused by hypertension, hypertensive cerebral vascular disease, rupture of aneurysm, an embolus, a thrombus, an angioma, blood dyscrasias, cardiac failure, systemic hypotension, cardiac arrest, cardiogenic shock, septic shock, spinal cord trauma, head trauma, seizure, bleeding from a tumor, or other blood loss.


SEQUENCE LISTING (5 sequences)

SEQ ID NO: 1 - DNA, 3836 bp, rattus rattus
  CDS (22)..(1569)
tacccgggggcgcgccgcaccatggagcccgccgtgtcgctggccgtgtgcgcgctgctctttctgctctgggtgcgagtgaaggggttggagttcgttctcatccaccaacgctgggtgtttgtgtgcctcttcctgctaccgctctccctcatcttcgacatctactactacgtgcgcgcctgggtggtgttcaagctgagcagtgcgccgcgcttgcacgagcagcgcgtgcaggacatccagaaacaggtacgggagtggaaggagcagggcagtaagaccttcatgtgcacggggcgtcctggatggctcactgtctcactgcgggttgggaagtacaagaagacgcataagaacatcatgatcaacctgatggacattctggaggtggacaccaagaaacagattgtgcgagtggagcccttggtgtctatgggtcaggtgacagctttgctgaactccattggctggactctgcctgtgttgcctgaactcgatgacctcacagtggggggcctgatcatgggcaccggcatcgagtcatcgtcccacaagtatggcctgttccaacacatctgtacggcctacgagctaatcctggcagatggcagctttgtgcgctgcacaccgtctgaaaactcagacctgttctatgccgtaccctggtcctgtgggaccctgggcttcctggtggctgctgagatccgcatcatccctgccaagaagtatgtcaagctgcggtttgagccagtgcggggcctggaggccatctgtgagaaattcacccatgagtcccagcggctggagaaccacttcgtggaagggttgctgtattccttggatgaggctgtcatcatgacaggggtcatgacggatgatgtagagcccagcaagctgaatagcattggcagttactacaagccgtggttcttcaagcatgtggagaactacctgaagacaaaccgagagggccttgaatacattcccctgaggcactactaccaccgscacacgcgcagcatcttctgggagctccaggacatcatcccttttggcaacaaccccatcttccgctacctctttggctggatggtgcctcccaagatctccctcctgaagctgacccagggcgagactctgcgtaaactgtatgaacagcttcatgtggtacaggacatgctggtgcccatgaaatgcctgtctcaggccctgcataccttccaaaatgacatccacgtctaccccatctggctgtgcccgttcatcctgcccagccagccaggacttgtgcatcccaagggagatgaggctgaactctacgtggacatcggggcatatggggagccacgggtgaagcacttcgaggccaggtcctgcatgaggcagctggagaagtttgtgcggagcgtgcacgggttccagatgttatacgccgattgctatatgaaccgggaggaattctgggagatgttcgatggctccttgtaccacaagctgcgcaagcagcttggctgccaggacgccttccctgaggtgtatgacaagatctgcaaggctgcgcggcactgagcagggcccctggggagccagctgcatgggaatggactcgctctccctcgctccaacttctctgctttcccagatttcagagagaaaccccttcagaaatccccgaggtagcccgacggcctccagggcagctcaagcaagtggaagcacgtgggatttggagtcagacagagctaagtcccagtcccagattagccactcattagctgtgtgactgagtctgggctagtcacctgaccctgtgtgccccgtgttcatctgtgaaaggaggtataaatccctacctgctggacgtcattcacacaaagggcccatcgtgtgggaggggcttcgtaagtggtagccaacaccgctgcttccatgtaacctcacacccggctcatgctttggattcaacagctgagtccagacaggttccccaccggtcacctgtgcacgggtttggctggcaccatgaaggggattccttggagtttcacctgcataacctgcatcagccccaagagggccttcccctgggtctcggggattgagggtcatctgattggcaggcaggcccaggatgtgctgtgccaaagccaggtctgctccaaagtgctctctgccatcttcggaccgtcctgcccctacttgtgtgctcaggccctcgggccaccccagccatcactgagcccactcaagactgttcctggaagacatctcaagatgggaagcctagccttcctgggctttcctttcttgacttccggcatccaggcctcctctagagtccactgagtcacaatgggggcacatgctcatgactgtgtcacctaggggtggttcccacactgagtgagaaaggggcaaccgccctgttgttactgtgaggaccctccctggggttggagagaaagcatccagggtcactcatccccctctcccatttcgttcccactcccccggctctagttaatttcagtgccttacaaatcctaagctcagaaaaagttagctccatttccgttcctgagggaggggcctctgaggtcctcctgccttgttaaagaagtgtggttggttgtgcagcccccaccttcagaactgcctggcccagctgagagcccagagtctgagggggaatcaagttctggaaggaaagccagaactcaagcactgagctggttgcagtctcccccaccccgccacccctgctccccgctccaaaagctgcaattttctcacccgtcatgtcggaacataagagctcctgaaacctttctaccagatggcggttagggaccgttcatcagtagcccttccctcctccaccaagccagctcccatccatgattttcatccatctggcaagggccagaacccttagagtcatagtcgaaggatgctcactgatgctgctgtgggggtggggatcctggtcagtgctactgcagtcctgtgaaggccggtgcactcggggcaagttggaaggcagaagaatcacctcatccatttgacctgtgaaggaagtaacctcactttgctgtctgagcagcatgcgttcctggcatgggctctcctcgtggtccgtgtgcatccttgcgctcgcagaccgatgaccctgcttgctgcaggtgactggtcaagtgcgagctagcttagttagtgtggtgtataaggcgccatcattcctccagtaagcctccatcccaaagcaactgaggctgtggcagtgatgccagcaacctgtgtcacccaaaattatccagccctccacgggcactgcctaggacctggggagggaagggactttgcatcacagtagcctcaggttcgtgtttggctctggtaatatcttgcctgaaaagtggtggtcttccaggcatggtgtacggggtcccagccttgtcttagagcctgtcctgtcatcatccagccacgcagtggcttggtggagtcactttgctcctgtgggacagtgcgtgtaggaagtcaatgcaaagcctctccccttcatggctctggtttcttggctgcatggttctctgtaaatccagttatagccactgtctggctagaaagctgaattggctgatgctgaaagggaagagggggatgggtgtggcagtgtgtgctgtgtgatattctttaatttggtcatggggaccaaggagaaggcatgaattccccttgtcaggctcctgcagccttgggcactgtgcctactctccagaacacgtccctgtggctctggactgtttaaacctgcaggcatgcaagctatc

SEQ ID NO: 2 - protein, 516 aa, rattus rattus
MEPAVSLAVCALLFLLWVRVKGLEFVLIHQRWVFVCLFLLPLSLIFDIYYYVRAWVVFKLSSAPRLHEQRVQDIQKQVREWKEQGSKTFMCTGRPGWLTVSLRVGKYKKTHKNIMINLMDILEVDTKKQIVRVEPLVSMGQVTALLNSIGWTLPVLPELDDLTVGGLIMGTGIESSSHKYGLFQHICTAYELILADGSFVRCTPSENSDLFYAVPWSCGTLGFLVAAEIRIIPAKKYVKLRFEPVRGLEAICEKFTHESQRLENHFVEGLLYSLDEAVIMTGVMTDDVEPSKLNSIGSYYKPWFFKHVENYLKTNREGLEYIPLRHYYHRHTRSIFWELQDIIPFGNNPIFRYLFGWMVPPKISLLKLTQGETLRKLYEQLHVVQDMLVPMKCLSQALHTFQNDIHVYPIWLCPFILPSQPGLVHPKGDEAELYVDIGAYGEPRVKHFEARSCMRQLEKFVRSVHGFQMLYADCYMNREEFWEMFDGSLYHKLRKQLGCQDAFPEVYDKICKAARH

SEQ ID NO: 3 - DNA, 4094 bp, Homo sapiens
  CDS (37)..(1584)
cgcgaacccgcagggtacccgggggcgcgccgcaccatggagcccgccgtgtcgctggccgtgtgcgcgctgctcttcctgctgtgggtgcgcctgaaggggctggagttcgtgctcatccaccagcgctgggtgttcgtgtgcctcttcctcctgccgctctcgcttatcttcgatatctactactacgtgcgcgcctgggtggtgttcaagctcagcagcgctccgcgcctgcacgagcagcgcgtgcgggacatccagaagcaggtgcgggaatggaaggagcagggtggcaagaccttcatgtgcacggggcgccctggctggctcactgtctcactacgtgtcgggaagtacaagaagacacacaaaaacatcatgatcaaccagatggacattctggaagtggacaccaagaaacagattgtccgtgtggagcccttggtgaccatgggccaggtgactgccctgctgacctccattggctggactctccccgtgttgcctgagcttgatgacctcacagtggggggcttgatcatgggcacaggcatcgagtcatcatcccacaagtacggcctgttccaacacatctgcactgcttacgagctggtcctggctgatggcagctttgtgcgatgcactccgtccgaaaactcagacctgttctatgccgtaccctggtcctgtgggacgctgggtttcctggtggccgctgagatccgcatcatccctgccaagaagtacgtcaagctgcgtttcgagccagtgcggggcctggaggctatctgtgccaagttcacccacgagtcccagcggcaggagaaccacttcgtggaagggctgctctactccctggatgaggctgtcattatgacaggggtcatgacagatgaggcagagcccagcaagctgaatagcattggcaattactacaagccgtggttctttaagcatgtggagaactatctgaagacaaaccgagagggcctggagtacattcccttgagacactactaccaccgccacacgcgcagcatcttctgggagctccaggacatcatcccctttggcaacaaccccatcttccgctacctctttggctggatggtgcctcccaagatctccctcctgaagctgacccagggtgagaccctgcgcaagctgtacgagcagcaccacgtggtgcaggacatgctggtgcccatgaagtgcctgcagcaggccctgcacaccttccaaaacgacatccacgtctaccccatctggctgtgtccgttcatcctgcccagccagccaggcctagtgcaccccaaaggaaatgaggcagagctctacatcgacattggagcatatggggagccgcgtgtgaaacactttgaagccaggtcctgcatgaggcagctggagaagtttgtccgcagcgtgcatggcttccagatgctgtatgccgactgctacatgaaccgggaggagttctgggagatgtttgatagctccttgtaccacaagctgcgagagaagctgggttgccaggacgccttccccgaggtgtacgacaagatctgcaaggccgccaggcactgagctggagcccgcctggagagacagacacgtgtgagtggtcaggcatcttcccttcactcaagcttggctgctttcctagatccacactttcaaagagaaacccctccagaactcccaccctgacagcccaacaccaccttcctcctggcttccagggggcagcccagtggaatggaaagaatgtgggatttggagtcagacaagcctgagtccagttccccgtttagaactcattagctgtgtgactctgggtgagtcccttaacccctctgagcccgggtctcttcattagttgaaagggatagtaatacctacttgcaggttgttgtcatctgagttgagcactggtcacattgaaggtgctgggtaagtggtagctcttgttgcttcccgttcagcgtcacatctgcagtggagcctggaaaggctccacattaggtcacctgtgcacagccatggctggaatgatgaaggggatacgctggagttgccctgccatcgcctccatcagccagacgaggtcctcacaggagaaggacagctcttccccaccctgggatctcaggagggcagccacggagtggggaggccccagatgcgctgtgccaaagccaggtccgaggccaaagttctccctgccatccttggtgccgtcctgccccttcctccttcatgcctgggcctgcaggcccaccccagccaccactgagtccactcggagtgccctgtgttcctggagaaggcattccagggttgaatcttgtcccagcctcagcctgggacacctaggtggagagagtggtctccgctctgaattggatccaggggacctgggctcattcttcttggctcaccaaccctgcaggcctcatctttcccaaaacccactttgtcttggtgggagtgggtccgcgctgctctgcagcaggggctggggagtggacagcatcaggtgggaaagtggagtccaccctcatgtttctgtaggattctcaccgtggggctggaagaaaagagcatcgacttgatttctccaaccactcatccctctttttctttcttccaccactccccaccccagctgtagttaatttcagtgccttacaaatcctaagctcagagaaagttccatttccgttccagagggaagggaacctccctaggtccttccctggcttgttataacgcaaagcttggttgtttatgcaactctatcttaagaactgcccagcctcagttggaaacccgaatctgagaaggaattgcgtcatgtaagggaagctggaattaagggagctgagccagtcatggttgcggcgtgtgagtcaggagacctaggtttcagcccctctctactgtcagcgagctgtgcaacgtgggcaagtcattgtcctctgagctgcagtttcctcatctgtcacatcgctacagacaagacctccctggaacccttctgattgtcttggacactgtggttgcaaaacccacggaaagcctcatttttgtggaaagtcagaggaaaaatgatccagtggacacttggggattatctgtcattcaagatccttccttcaaccccaaggtcagctcccatctcatttccagaaaggctcatacctggcttgcagggaagcatctgtcttgtcattccaggtgccagaatcctctcagagtcattgaagggtgttcacccaccccgcccaaggcttggcacactgccagtgtcttagcagggtcttgtgagggctgggggcatccaggcactcagaaggcaaaggaaccaccctacccatttggcctctggagggggcagaagaaagaaagaaacctcatcctatattttacaaagcatgtgaattctggcattagctctcataggagacccatgtgcttccttgctcagtgcaaaactgatgattctacttgctgtagatgaatggttaacacgagctagttaaacagtgccattgttttgccagtgaagcctccaaccctaagccactgggacggtggccagagatgccagcagcctctgtcgcccttagtcatataaccaaaatccagaccttatccacaacccggggcttggaaaggaaggtattttggaatcacaccctccggttatgttgctccagtaaaatcttgcctggaaagaggcagtcttcttagcatggtgagctgagttcatggcttttttttgtagccagtcctgtccctggccatccatgtgatggttttggatggagttaaacttgatgccagtgggcagtgcatgtggaaagtatcagagtaaggctctcccctccagagccctgagtttcttggctgcatgaaggttttctttagaatcagaattgtagccagtttctttggccagaaggatgaatacttggatattactgaaagggaggggtggagatgggtgtggcagtgtatggtgtgtgatttttattttcttctttggtcatgggggccaaggagaaaggcatgaatcttccctgtcaggc

SEQ ID NO: 4 - protein, 516 aa, Homo sapiens
MEPAVSLAVCALLFLLWVRLKGLEFVLIHQRWVFVCLFLLPLSLIFDIYYYVRAWVVFKLSSAPRLHEQRVRDIQKQVREWKEQGGKTFMCTGRPGWLTVSLRVGKYKKTHKNIMINQMDILEVDTKKQIVRVEPLVTMGQVTALLTSIGWTLPVLPELDDLTVGGLIMGTGIESSSHKYGLFQHICTAYELVLADGSFVRCTPSENSDLFYAVPWSCGTLGFLVAAEIRIIPAKKYVKLRFEPVRGLEAICAKFTHESQRQENHFVEGLLYSLDEAVIMTGVMTDEAEPSKLNSIGNYYKPWFFKHVENYLKTNREGLEYIPLRHYYHRHTRSIFWELQDIIPFGNNPIFRYLFGWMVPPKISLLKLTQGETLRKLYEQHHVVQDMLVPMKCLQQALHTFQNDIHVYPIWLCPFILPSQPGLVHPKGNEAELYIDIGAYGEPRVKHFEARSCMRQLEKFVRSVHGFQMLYADCYMNREEFWEMFDSSLYHKLREKLGCQDAFPEVYDKICKAARH

SEQ ID NO: 5 - DNA, 312 bp, Mus sp.
atggagcccgccgtgtcgctggccgtgtgcgcgctgctctttctgctctgggtgcgagtgaaggggttggagttcgttctcatccaccagcgctgggtgttcgtgtgcctcttcttgctgccgctctcgctcatcttcgatatctactactacgtgcgcgcctgggtggtgttcaagctgagcagtgcgccgcgcctgcacgagcagcggtgcgggacatccagaaacaggtccgggaatggaaggaacagggcagtaagaccttcatgtgcacggggcgcccaggctggctcactgtctcgctgcgagtcg